SEQUENCE LISTING (3 sequences)

SEQ ID NO: 1 - protein, 1935 aa, Mus musculus
MADAEMAAFGAAAPFLRKSEKERLEAQTRPFDLKKDVFVPDDKEEFVKAKIVSREGGKVTAETENGKTVTVKEDQVMQQNPPKFDKIEDMAMLTFLHEPAVLYNLKERYASWMIYTYSGLFCVTVNPYKWLPVYNAEVVAAYRGKKRSEAPPHIFSISDNAYQYMLTDRENQSILITGESGAGKTVNTKRVIQYFAVIAAIGDRSKKDQTPGKGTLEDQIIQANPALEAFGNAKTVRNDNSSRFGKFIRIHFGATGKLASADIETYLLEKSRVIFQLKAERDYHIFYQILSNKKPELLDMLLITNNPYDYAFISQGETTVASIDDSEELMATDSAFDVLGFTPEEKNSIYKLTGAIMHFGNMKFKQKQREEQAEPDGTEEADKSAYLMGLNSADLLKGLCHPRVKVGNEYVTKGQNVQQVSYAIGALAKSVYEKMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLYDNHLGKSNNFQKPRNVKGKQEAHFSLVHYAGTVDYNILGWLQKNKDPLNETVVGLYQKSSLKLLSNLFANYAGADAPADKGKGKAKKGSSFQTVSALHRENLNKLMTNLRSTHPHFVRCIIPNETKSPGVMDNPLVMHQLRCNGVLEGIRICRKGFPNRILYGDFRQRYRILNPAAIPEGQFIDSRKGAEKLLGSLDIDHNQYKFGHTKVFFKAGLLGLLEEMRDERLSRIITRIQAQSRGVLSRMEFKKLLERRDSLLIIQWNIRAFMGVKNWPWMKLYFKIKPLLKSAETEKEMATMKEEFGRVKDALEKSEARRKELEEKMVSLLQEKNDLQLQVQAEQDNLADAEERCDQLIKNKIQLEAKVKEMTERLEDEEEMNAELTAKKRKLEDECSELKRDIDDLELTLAKVEKEKHATENKVKNLTEEMAGLDEIIVKLTKEKKALQEAHQQALDDLQAEEDKVNTLTKAKVKLEQQVDDLEGSLEQEKKVRMDLERAKRKLEGDLKLTQESIMDLENDKQQLDERLKKKDFELNALNARIEDEQALGSQLQKKLKELQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKMCRTLEDQMNEHRSKAEETQRSVNDLTSQRAKLQTENGELSRQLDEKEALISQLTRGKLTYTQQLEDLKRQLEEEVKAKNALAHALQSARHDCDLLREQYEEETEAKAELQRVLSKANSEVAQWRTKYETDAIQRTEELEEAKKKLAQRLQDAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKPENKNLQEEISDLTEQLGSTGKSIHELEKIRKQLEAEKLELQSALEEAEASLEHEEGKILRAQLEFNQIKAEIERKLAEKDEEMEQAKRNHLRMVDSLQTSLDAETRSRNEALRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMDADLSQLQTEVEEAVQECRNAEEKAKKAITDAAMMAEELKKEQDTSAHLERMKKNMEQTIKDLQHRLDEAEQIALKGGKKQLQKLEARVRELENELEAEQKRNAESVKGMRKSERRIKELTYQTEEDRKNLLRLQDLVDKLQLKVKAYKRQAEEAEEQANTNLSKFRKVQHELDEAEERADIAESQVNKLRAKSRDIGAKGLNEE

SEQ ID NO: 2 - protein, 1935 aa, Mus musculus
MADAEMAAFGAAAPFLRKSEKERLEAQTRPFDLKKDVFVPDDKEEFVKAKIVSREGGKVTAETENGKTVTVKEDQVMQQNPPKFDKIEDMAMLTFLHEPAVLYNLKERYASWMIYTYSGLFCVTVNPYKWLPVYNAEVVAAYRGKKRSEAPPHIFSISDNAYQYMLTDRENQSILITGESGAGKTVNTKRVIQYFAVIAAIGDRSKKDQTPGKGTLEDQIIQANPALEAFGNAKTVRNDNSSRFGKFIRIHFGATGKLASADIETYLLEKSRVIFQLKAERDYHIFYQILSNKKPELLDMLLITNNPYDYAFISQGETTVASIDDSEELMATDSAFDVLGFTPEEKNSIYKLTGAIMHFGNMKFKQKQREEQAEPDGTEEADKSAYLMGLNSADLLKGLCHPRVKVGNEYVTKGQNVQQVSYAIGALAKSVYEKMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLYDNHLGKSNNFQKPRNVKGKQEAHFSLVHYAGTVDYNILGWLQKNKDPLNETVVGLYQKSSLKLLSNLFANYAGADAPADKGKGKAKKGSSFQTVSALHRENLNKLMTNLRSTHPHFVRCIIPNETKSPGVMDNPLVMHQLRCNGVLEGIRICRKGFPNRILYGDFRQRYRILNPAAIPEGQFIDSRKGAEKLLGSLDIDHNQYKFGHTKVFFKAGLLGLLEEMRDERLSRIITRIQAQSRGVLSRMEFKKLLERRDSLLIIQWNIRAFMGVKNWPWMKLYFKIKPLLKSAETEKEMATMKEEFGRVKDALEKSEARRKELEEKMVSLLQEKNDLQLQVQAEQDNLADAEERCDQLIKNKIQLEAKVKEMTERLEDEEEMNAELTAKKRKLEDECSELKRDIDDLELTLAKVEKEKHATENKVKNLTEEMAGLDEIIVKLTKEKKALQEAHQQALDDLQAEEDKVNTLTKAKVKLEQQVDDLEGSLEQEKKVRMDLERAKRKLEGDLKLTQESIMDLENDKQQLDERLKKKDFELNALNARIEDEQALGSQLQKKLKELQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKMCRTLEDQMNEHRSKAEETQRSVNDLTSQRAKLQTENGELSRQLDEKEALISQLTRGKLTYTQQLEDLKRQLEEEVKAKNALAHALQSARHDCDLLREQYEEETEAKAELQRVLSKANSEVAQWRTKYETDAIQRTEELEEAKKKLAQRLQDAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGSTGKSIHELEKIRKQLEAEKLELQSALEEAEASLEHEEGKILRAQLEFNQIKAEIERKLAEKDEEMEQAKRNHLRMVDSLQTSLDAETRSRNEALRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMDADLSQLQTEVEEAVQECRNAEEKAKKAITDAAMMAEELKKEQDTSAHLERMKKNMEQTIKDLQHRLDEAEQIALKGGKKQLQKLEARVRELENELEAEQKRNAESVKGMRKSERRIKELTYQTEEDRKNLLRLQDLVDKLQLKVKAYKRQAEEAEEQANTNLSKFRKVQHELDEAEERADIAESQVNKLRAKSRDIGAKGLNEE

SEQ ID NO: 3 - protein, 1935 aa, Homo sapiens
MGDSEMAVFGAAAPYLRKSEKERLEAQTRPFDLKKDVFVPDDKQEFVKAKIVSREGGKVTAETEYGKTVTVKEDQVMQQNPPKFDKIEDMAMLTFLHEPAVLYNLKDRYGSWMIYTYSGLFCVTVNPYKWLPVYTPEVVAAYRGKKRSEAPPHIFSISDNAYQYMLTDRENQSILITGESGAGKTVNTKRVIQYFAVIAAIGDRSKKDQSPGKGTLEDQIIQANPALEAFGNAKTVRNDNSSRFGKFIRIHFGATGKLASADIETYLLEKSRVIFQLKAERDYHIFYQILSNKKPELLDMLLITNNPYDYAFISQGETTVASIDDAEELMATDNAFDVLGFTSEEKNSMYKLTGAIMHFGNMKFKLKQREEQAEPDGTEEADKSAYLMGLNSADLLKGLCHPRVKVGNEYVTKGQNVQQVIYATGALAKAVYERMFNWMVTRINATLETKQPRQYFIGVLDIAGFEIFDFNSFEQLCINFTNEKLQQFFNHHMFVLEQEEYKKEGIEWTFIDFGMDLQACIDLIEKPMGIMSILEEECMFPKATDMTFKAKLFDNHLGKSANFQKPRNIKGKPEAHFSLIHYAGIVDYNIIGWLQKNKDPLNETVVGLYQKSSLKLLSTLFANYAGADAPIEKGKGKAKKGSSFQTVSALHRENLNKLMTNLRSTHPHFVRCIIPNETKSPGVMDNPLVMHQLRCNGVLEGIRICRKGFPNRILYGDFRQRYRILNPAAIPEGQFIDSRKGAEKLLSSLDIDHNQYKFGHTKVFFKAGLLGLLEEMRDERLSRIITRIQAQSRGVLARMEYKKLLERRDSLLVIQWNIRAFMGVKNWPWMKLYFKIKPLLKSAEREKEMASMKEEFTRLKEALEKSEARRKELEEKMVSLLQEKNDLQLQVQAEQDNLADAEERCDQLIKNKIQLEAKVKEMNERLEDEEEMNAELTAKKRKLEDECSELKRDIDDLELTLAKVEKEKHATENKVKNLTEEMAGLDEIIAKLTKEKKALQEAHQQALDDLQAEEDKVNTLTKAKVKLEQQVDDLEGSLEQEKKVRMDLERAKRKLEGDLKLTQESIMDLENDKQQLDERLKKKDFELNALNARIEDEQALGSQLQKKLKELQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKMCRTLEDQMNEHRSKAEETQRSVNDLTSQRAKLQTENGELSRQLDEKEALISQLTRGKLTYTQQLEDLKRQLEEEVKAKNALAHALQSARHDCDLLREQYEEETEAKAELQRVLSKANSEVAQWRTKYETDAIQRTEELEEAKKKLAQRLQEAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGSSGKTIHELEKVRKQLEAEKMELQSALEEAEASLEHEEGKILRAQLEFNQIKAEIERKLAEKDEEMEQAKRNHLRVVDSLQTSLDAETRSRNEALRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMDADLSQLQTEVEEAVQECRNAEEKAKKAITDAAMMAEELKKEQDTSAHLERMKKNMEQTIKDLQHRLDEAEQIALKGGKKQLQKLEARVRELENELEAEQKRNAESVKGMRKSERRIKELTYQTEEDRKNLLRLQDLVDKLQLKVKAYKRQAEEAEEQANTNLSKFRKVQHELDEAEERADIAESQVNKLRAKSRDIGTKGLNEE

(19) United States
(12) Patent Application Publication
(10) Pub. No.: US 2022/0030837 A1
(43) Pub. Date: Feb. 3, 2022

(54) Novel Animal Model For Laing Distal Myopathy (Mpd1) And Methods of Use Thereof

(71) Applicant: The Regents of the University of Colorado, a body corporate, Denver, CO (US)

(72) Inventors: Leslie Leinwand, Boulder, CO (US); Massimo Buvoli, Boulder, CO (US); Ada Buvoli, Boulder, CO (US); Genevieve Wilson, Arvada, CO (US)

(21) Appl. No.: 17/392,641

(22) Filed: Aug. 3, 2021

Related U.S. Application Data
(60) Provisional application No. 63/060,374, filed on Aug. 3, 2020.

(51) Int. Cl. A01K 67/027; C12N 15/85

(52) U.S. Cl. CPC A01K 67/0276; C12N 15/8509; C12N 2015/8527; A01K 2227/105; A01K 2267/03; A01K 2217/052

(57) ABSTRACT

The inventive technology is directed to the generation of a novel transgenic mammalian model for the study of Laing distal myopathy. The novel animal model of the invention may include a transgenic animal, and preferably a transgenic mouse, expressing the β-myosin R1500P mutation transgene that produces one or more phenotypes associated with MPD1. The β-myosin R1500P mutation transgene may further be selectively expressed in fast muscle tissue of the transgenic animal.

### CROSS REFERENCES TO RELATED APPLICATION

This application claims the benefit of and priority to U.S. Provisional Application No. 63/060,374, filed Aug. 3, 2020. The entire specification and figures of the above-referenced application is hereby incorporated in its entirety by reference.

### GOVERNMENT INTEREST

This invention was made with government support under grant number GM029090 awarded by the National Institutes of Health. The government has certain rights in the invention.

### TECHNICAL FIELD

This inventive technology is generally directed to the fields of genetics, and the generation of transgenic animal models for the study of disease. In particular, the inventive technology is directed to the generation of a novel transgenic mammalian model for the study of Laing distal myopathy.

### BACKGROUND

Laing distal myopathy (MPD1) is an autosomal dominant disease with variable timing of disease onset that spans from birth into adulthood. MPD1 affects skeletal muscle function in a progressive manner: clinically, symptoms begin with weakness in the lower leg anterior compartment that impacts ankle and great toe dorsiflexion. Contrary to many other muscle disorders, pathologic findings in muscle biopsies from MPD1 patients are often inconsistent. MPD1-causing mutations have been mapped to the MYH7 gene (SEQ ID NO.3) that encodes the human β-myosin heavy chain (also generally referred to herein as “β-myosin” or “β-myosin gene”) the primary myosin motor expressed in both human heart and in type I, slow skeletal muscle fibers. Unexpectedly, only a small number of MPD1 patients also develop a cardiomyopathy, despite higher levels of β-myosin expression in the heart. Myosin is a hexameric molecule comprised of a pair of heavy chains and two pairs of non-identical light chains. ˜400 pathogenic mutations causing cardiac and skeletal myopathies have been identified in both the N-terminal motor domain as well as in the coiled-coil rod region of MYH7¹. The majority of MPD1 mutations are located in ¹ www.hgmd.cf.ac.uk/ac/index.php

the light meromyosin (LMM) domain corresponding to the C-terminal third of the rod that controls assembly of myosin into the thick filaments. However, a small number of them are also located in the motor domain. MPD1 mutations are primarily codon deletions and missense mutations that introduce a proline residue. Both of these genetic defects are predicted to negatively impact the structure of the myosin coiled-coil. For example, proline residues found in α-helices induce a ˜26-degree kink that could locally unwind the myosin coiled-coil.

The biological effects of a subset of MPD1 mutations have been characterized in both non-muscle and muscle cells. Muscle cell-based studies have shown that proline rod mutations do not impair incorporation of the mutant myosins into the sarcomere and therefore, do not block formation of the myosin thick filament as originally proposed. However, they can trigger myosin cytoplasmic aggregates or cause aberrant myosin packing in thick filaments. A progressive dominant hind/fore limb myopathy resembling MPD1 but associated with high frequency of myocardial infarctions has been reported in pigs. In this model, sequence analysis revealed an in-frame insertion of two residues (alanine, proline) in MYH7 exon 30; muscle fiber degeneration and regeneration and interstitial fibrosis were also observed. More recently, to characterize the molecular mechanisms of the MPD1-causing mutation L1729del, a Drosophila melanogaster model for MPD1 was established. By recapitulating some of the morphological muscle defects such as sarcomeric disorganization and myofibril damage observed in patients, this study provided new insight into the pathogenesis of the disease. However, how MDP1 rod mutations act in the mammalian muscle environment remains unclear and understudied. In fact, while numerous genetic mouse models have been developed for studying MYH7 motor domain mutations that cause hypertrophic or dilated cardiomyopathy, no mammalian genetic models have yet been reported for examining the effects of myopathy-causing mutations in the rod domain.

To address this long-felt need, the invention technology includes the first MPD1 mouse model expressing the R1500P rod mutation that causes MPD1 (SEQ ID NO. 1). As noted below, expression of the mutant myosin affects both muscle histological structure and performance. Further, transgenic mice expressing the R1500P rod mutation (generally referred to herein as “R1500P,” or “R1500P mutation,” or “βR1500P”) show decreased muscle strength and endurance, as well as decreased resistance to fatigue. Moreover, the presence of the R1500P rod mutation weakens actomyosin binding by affecting the cross-bridge detachment rate. Since the phenotype of the transgenic mice closely mimics MDP1, the novel animal model of the invention may be a useful platform for testing and developing future therapeutic interventions for MPD1.

### SUMMARY OF THE INVENTION

One aspect of the invention may include a novel animal model for the study of MPD1. In one preferred aspect, this novel animal model may include a transgenic animal, and preferably a mouse, expressing the β-myosin R1500P mutation transgene that produces one or more phenotypes associated with MPD1. In this preferred embodiment, the β-myosin R1500P mutation transgene may be selectively expressed in fast muscle tissue of the transgenic animal.

Another aspect of the invention includes a transgenic, non-human animal, or colony of animals, whose genome comprises a β-myosin R1500P mutant transgene wherein the arginine (R) residue at amino acid position 1500 is substituted with a proline (P) residue. In a preferred embodiment, the transgenic, non-human animal, or colony of animals may be a mouse, such as for example a Mus musculus.

Another aspect of the invention systems, and methods of producing a transgenic animal that has one or more phenotypes associated with MPD1. In one optional aspect, this method may include knocking-out, or disrupting expression of the wild-type β-myosin gene in an animal, and preferably a mouse, or in one or more tissues or organs of a mouse, and expressing a polynucleotide, operably linked to a promoter, encoding a β-myosin R1500P mutant transgene wherein the arginine (R) residue at amino acid position 1500 is substituted with a proline (P) residue, wherein the β-myosin R1500P mutant transgene produces one or more phenotypes associated with MPD1.

One aspect of the invention includes isolated polynucleotides and amino acid sequences for a β-myosin mutant transgene, and in particular a β-myosin R1500P mutant transgene. In one preferred aspect, the invention includes an expression vector, including a nucleotide sequence encoding the amino acid sequence according to SEQ ID NO. 1, or a fragment or variant thereof. operably linked to a promoter, and preferably a cell or tissue specific promoter. In another preferred aspect, this expression vector may be used to introduce the β-myosin R1500P mutant transgene to a cell.

Another aspect of the invention may include methods of screening therapeutic compounds, or other therapies for their effects on one or more pathological phenotypes associated MPD1. In one preferred aspect, the invention may include a method of determining the efficacy of a therapeutic compound for the treatment of MPD1 comprising the step of, administering a pharmaceutically effective amount of a therapeutic compound directed to the treatment of one or more pathological phenotypes associated MPD1 to the transgenic animal, and preferably a mouse expressing the β-myosin R1500P mutant transgene and determining if the therapeutic compound decreases one or more phenotypes associated with MPD1, and comparing any phenotype changes with an animal or mouse that did not receive the therapeutic compound or is not transgenic and expresses a wild-type β-myosin gene.

Additional aspects of the invention will become apparent from the figures and descriptions provided below.

### BRIEF DESCRIPTION OF THE FIGURES

FIG. 1A-E. Characterization of R1500P transgenic muscle. (A) Representative western blot analysis performed with tibialis anterior total protein extracts from the indicated genotypes using myc and α-sarcomeric actin (control) antibodies. (B) Quantification of transgenic myosin relative to total myosin in the tibialis anterior muscle. (C) Tibialis anterior muscle weight and muscle/body weight ratio of βWT and R1500P mice (n=20/group). (D) Representative SDH activity as shown by staining cross-sections of tibialis anterior muscle of βWT and R1500P mice. Quantification of percentage of SDH positive muscle fibers and muscle fiber size are also shown. (E) Relative mRNA expression levels of myosin heavy chain isoforms in the tibialis anterior muscle from βWT and R1500P mice (n=10/group). Data are expressed as mean+/−SEM. *p<0.05, ****p<0.0001 by two-tailed unpaired t-test with Welch's correction.

FIG. 2A-B. Tibialis anterior muscle shows ultrastructural changes in the SR and t-tubules in the presence of R1500P mutation. (A) In R1500P transgenic mice, t-tubules (black arrows) and sarcoplasmic reticulum (black arrowheads) abnormalities were shown by electron microscopy. βWT TA shows normal t-tubule triads with accompanying organized SR networks. R1500P TA shows dilated t-tubules with distended & enlarged SR. (B) Relative mitochondrial DNA content of CO1 normalized to 18S (n=4/group). Data are expressed as mean+/−SEM. *p<0.05 by two-tailed unpaired t-test with Welch's correction.

FIG. 3A-E. Activation of genes in the unfolded protein response (UPR) in R1500P transgenic mice. (A-E) Relative mRNA expression levels of PERK, ATF4, ATF3, GADD34, and CHOP in the tibialis anterior muscle from βWT and R1500P mice (n=8/group). Data are expressed as mean+/−SEM. *p<0.05, **p<0.01 by two-tailed unpaired t-test with Welch's correction.

FIG. 4A-C. Impaired muscle function in R1500P mutants. (A) Male βWT and R1500P mice were subjected to voluntary wheel running. Bars represent average running distance and speed over 28 days (n=10-12/group). (B) Four-limb hanging test recording the latency to when the animal falls. Average performance is the average of three trials (n=6/group). (C) Measurement of forelimb grip strength using computerized grip strength meter (n=6/group). Data are expressed as mean+/−SEM. *p<0.05, **p<0.01, ****p<0.0001 by two-tailed unpaired t-test with Welch's correction.

FIG. 5A-E. Altered contractility of intact skeletal muscle from R1500P TG mice. (A) Measurement of specific tetanic force in R1500P EDL compared to WT. (B) Measurement of specific twitch force in R1500P EDL compared to WT. (C) Ratio of twitch to tetanic force in βWT and R1500P EDL. (D) Force frequency curves for EDL muscles from βWT and R1500P muscles. Note the right-ward shift of the curve in TG EDL. (E) Percentage of force drop as EDL muscle relaxes after tetanic contraction, measured over 60 seconds (n=1/group). Data are expressed as mean+/−SEM. *p<0.05, **p<0.01 by two-tailed unpaired t-test with Welch's correction. Unless otherwise noted n=5/group (βWT), n=3/group (R1500P).

FIG. 6A-C. Weaker acto-myosin binding in presence of the R1500P mutation. Measured kinetics of relaxation from myofibrils were as follows: (A) Rate constant of early slow force decline. (B) Duration of early slow force decline. (C) Rate constant of the final exponential phase of force decline. Data are expressed as mean+/−SEM of 6-10 myofibrils/tibialis anterior muscle. *p<0.05 by two-tailed unpaired t-test with Welch's correction.

FIGS. 7A-B. Establishment of βWT and R1500P transgenic mouse lines. (A) Representative western blot analysis of transgene expression from established lines performed with tibialis anterior total protein extracts from the indicated genotypes using myc and a sarcomeric actin (control) antibodies. (B) Western blot analysis of transgene expression in different tissue types performed with total protein extracts from the indicated tissues using myc and α-sarcomeric actin (control) antibodies.

FIG. 8A-D. No change in contractility of intact soleus muscle from TG mice. (A) Measurement of specific tetanic force in R1500P soleus compared to βWT. (B) Measurement of specific twitch force in R1500P soleus compared to βWT. (C) Ratio of twitch to tetanic force in βWT and R1500P EDL. (D) Force frequency curves for soleus muscles from βWT and R1500P muscles. Data are expressed as mean+/−SEM.

FIG. 9A-D. R1500P mutation does not affect myofibril contractility activation. Measured activation kinetics from myofibrils were as follows: (A) The rate constant of tension development following maximal calcium activation. (B) The rate constant of tension redevelopment following a release-restretch applied to the activated myofibril. (C) Myofibril basal tension in fully relaxing condition. (D) Maximal tension generated at full calcium activation (pCa 4.5). Data are expressed as mean+/−SEM of 6-10 myofibrils/tibialis anterior muscle.

FIG. 10A-E. No effect on activation or relaxation kinetics in soleus muscle. Measured kinetics of relaxation and activation from myofibrils were as follows: (A) Rate constant of early slow force decline. (B) Duration of early slow force decline. (C) Rate constant of the final exponential phase of force decline. (D) The rate constant of tension development following maximal calcium activation. (E) The rate constant of tension redevelopment following a release-restretch applied to the activated myofibril. Data are expressed as mean+/−SEM of 6-10 myofibrils/soleus muscle.

### DETAILED DESCRIPTION OF THE INVENTION

Over 400 mutations in β-myosin have been identified in patients diagnosed with either cardiac or distal skeletal myopathy. A subset of these mutations is known to lead to Laing distal myopathy (MPD1) with mutations in β-myosin being the only known cause of MPD1. However, while this disease has previously been studied in a variety of systems, it is still not understood how these mutations lead to disease—particularly in a mammalian background. To address this long-felt need, the inventive technology included herein included the generation of the first mouse model for a mutation in the rod domain of β-myosin.

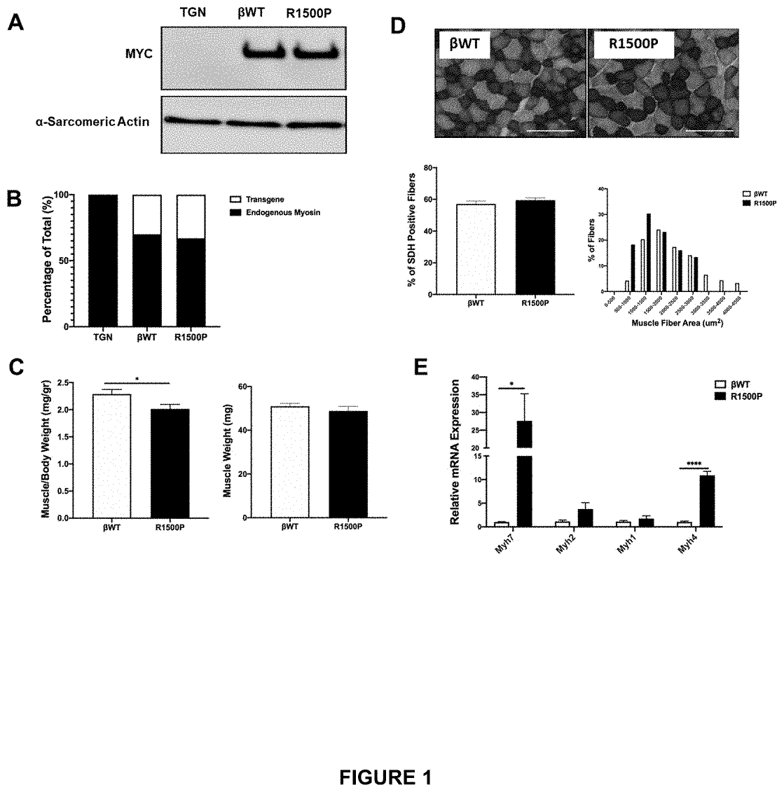

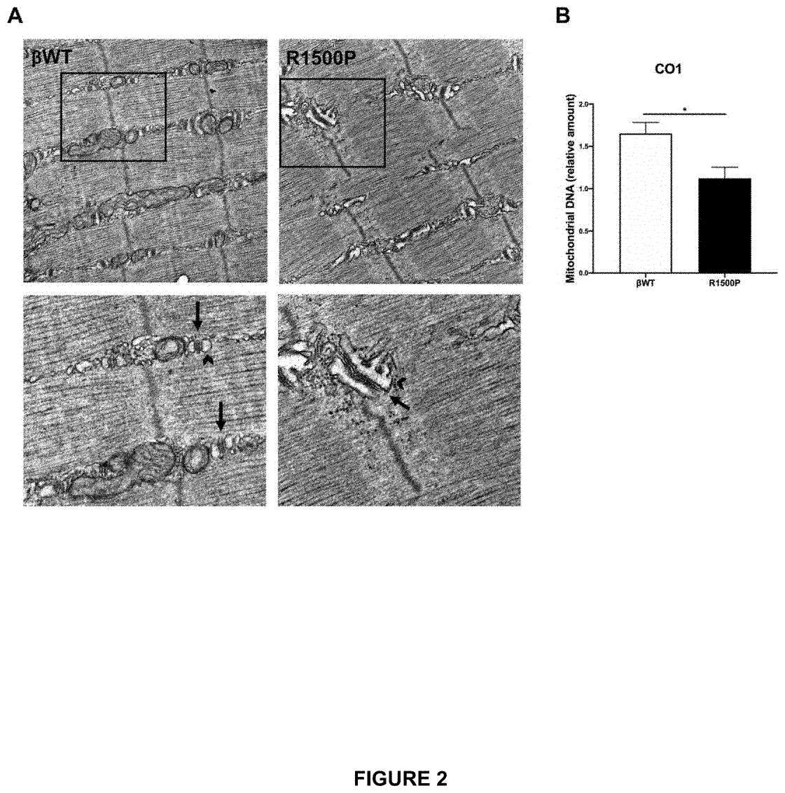

Patients diagnosed with Laing distal myopathy are known to present with variable muscle pathological changes, which might be due to the position of the mutation within the 0-myosin protein—with different mutations resulting in clinical variation. In patients with the R1500P mutation, hypotrophy of slow-type muscle fibers, but variable predominance of type 1 muscle fibers has been noted. Histological analyses of muscle from βR1500P mouse muscles shows recapitulation of this phenotype (FIG. 1D). While ultrastructural analysis has not previously been performed on patients with the R1500P mutation, the MPD1-causing K1729del has been shown to cause myofibrillar disorganization and mitochondrial abnormalities. No sarcomeric disorganization was noted in βR1500P transgenic animals; however, structural abnormalities were noted in the sarcoplasmic reticulum, t-tubules, and mitochondria (FIG. 2). Recapitulation of Laing distal histological hallmarks in βR1500P transgenic mice suggests that the novel animal model of the invention is a useful tool for studying the MPD1 and the underlying pathology.

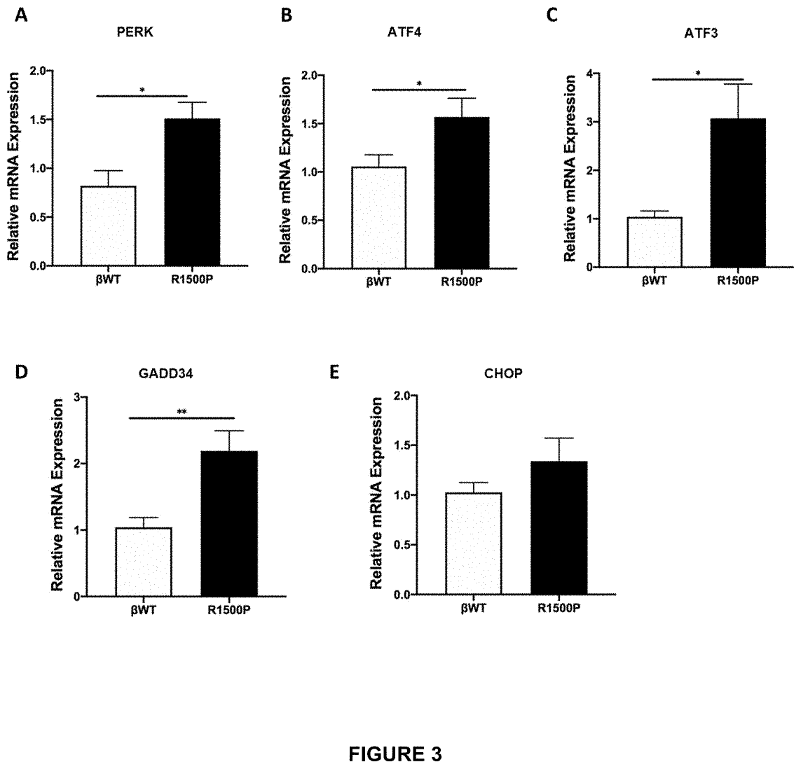

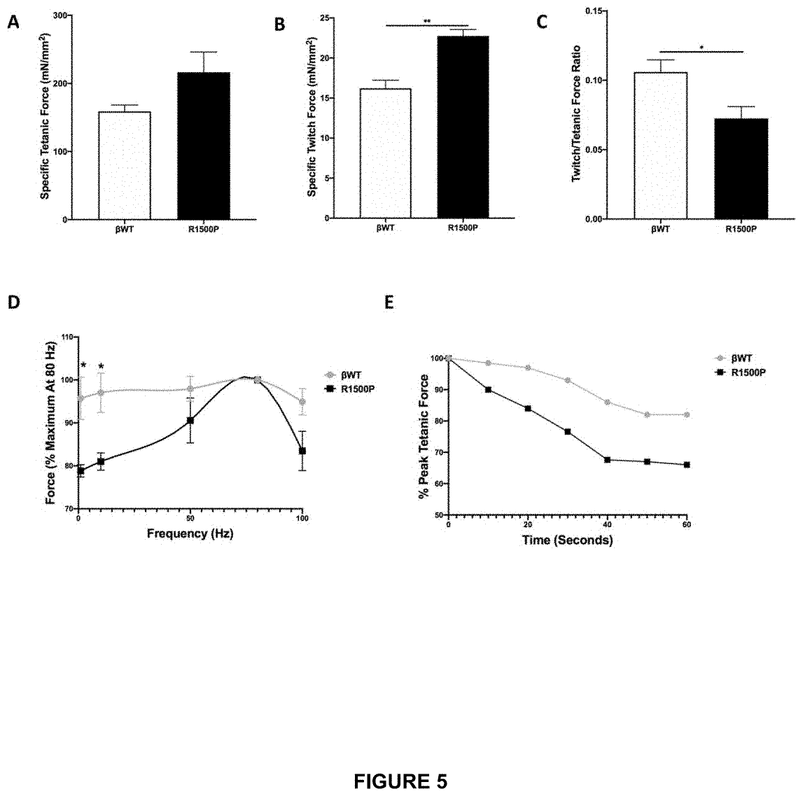

It was previously proposed that introduction of a proline into the α-helical strands of the myosin rod domain would affect proper assembly of the thick filament due to steric hindrance and the inability to form hydrogen bonds. In spite of the predicted effect, previous studies showed that muscle cells transfected with the mutation had organized sarcomeres indicating that the mutant myosin is able to be effectively incorporated into the thick filament—similar to WT. However, there are no data about the effect of this mutation on a functioning muscle. Interestingly, as shown in FIG. 3, mRNAs encoding PERK itself and some downstream effectors were activated, implicating ER stress and the unfolded protein response to the R1500P mutant myosin. These results are consistent with structural effects caused by the introduction of a proline residue. While this indicates that the mutant is not as well tolerated as WT myosin and may be contributing to the mechanical phenotypes observed (FIGS. 5,6), it is also likely that thick filaments containing MPD1 mutants are subject to increased sarcomeric turnover which can be assessed in future experiments.

The novel animal model of the invention showed that the presence of the R1500P mutation in fast-type skeletal muscle of a transgenic animal led to functional phenotypes, ultrastructural abnormalities in the SR and t-tubules, and contractile deficiencies—on the whole muscle level and at the level of the myofibril. Collectively, the observed phenotypes have all previously been linked to diminished muscle function, effects on cross-bridge detachment rate, and effects on calcium (Ca2+) handling. SR & t-tubule enlargement and disorganization have also been shown to play a role in decreased muscle strength and muscle atrophy. Furthermore, force production was shown to be impaired at low frequencies which can lead to insufficient Ca2+ release preventing full cross-bridge interaction to occur.

In one preferred embodiment, the invention may include methods of generating a transgenic animal. In this embodiment, an animal, and preferably a rodent or mouse may be genetic engineered to modify the expression of the wild-type β-myosin protein (SEQ ID NO. 2) and this modification may include optionally knocking-out or disrupting expression of the wild-type β-myosin gene in the animal, or in one or more tissues or organs of an animal, and preferably a mouse. As discussed below, genes may be knocked-out, or disrupted by various methods, such as insertion, deletion, substitution, and/or recombination. The transgenic animal, and gain, preferably a rodent or mouse, may be genetically engineered to express a β-myosin R1500P mutant transgene (SEQ ID NO. 1) wherein the arginine (R) residue at amino acid position 1500 is substituted with a proline (P) residue, or a fragment or variant thereof. Methods of producing transgenic mice are generally described below, and would be known and readily understood by one or ordinary skill in the art.

Expression of the β-myosin R1500P mutant transgene (SEQ ID NO. 1) may be under the control of a promoter, and more preferably a tissue-specific promoter. For example, in this preferred embodiment, expression of the β-myosin R1500P mutant transgene (SEQ ID NO. 1) may be localized to the animal's fast skeletal muscle fibers. In this preferred embodiment, a nucleotide sequence encoding the β-myosin R1500P mutant transgene (SEQ ID NO. 1) may be operably linked with a tissue-specific promoter, and preferably a muscle creatine kinase (MCK) promoter. This nucleotide sequence may be part of an expression vector, or other system to allow the introduction of the β-myosin R1500P mutant transgene (SEQ ID NO. 1) to the subject animal, and preferably the stable integration and expression of the β-myosin R1500P mutant transgene (SEQ ID NO. 1) operably linked to a muscle creatine kinase (MCK) promoter in the fast skeletal muscle fibers of a mouse, wherein the transgenic mouse exhibits at least one phenotype associated with MPD1. Additional embodiment may include a tag, such as a myc-tag coupled to the 3-myosin R1500P mutant transgene (SEQ ID NO. 1) to allow the presence and activity of the protein in the transgenic animal to be better tracked and studied. In this embodiment, the myc-tag comprises an eleven (11) amino acid residues and may substitute for the last 6 amino acids of the MYH7 (KGLNEE) which may be deleted.

As noted above, a transgenic, non-human animal of the invention expressing the β-myosin R1500P mutant transgene (SEQ ID NO. 1) may exhibit at least one phenotype associated with MPD1. In this preferred embodiment, a transgenic, rodent or mouse expressing the β-myosin R1500P mutant transgene (SEQ ID NO. 1) may exhibit one or more phenotype of MPD1 associated is selected from the group consisting of: abnormal muscle tissue or muscle atrophy; decreased the muscle/body weight ratio; muscle tissue had a higher proportion of smaller muscle fibers; upregulation of expression of myosin isoforms Myh7 and Myh4; abnormalities in sarcoplasmic reticulum (SR); abnormalities in t-tubules; abnormalities in mitochondria; upregulation of one or more gene of the unfolded protein response (UPR) pathway; upregulation of one or more gene of the PERK, or genes involved in the PERK pathway; upregulation of ATF4; upregulation of ATF3; upregulation of GADD34; decreased muscle strength; decreased resistance to fatigue; and weakened actomyosin binding, or a combination of the same.

Another embodiment of the invention includes the creation of a novel, Laing distal myopathy (MPD1) model animal, and preferably a rodent or mouse, engineered to express a 3-myosin R1500P mutant transgene (SEQ ID NO. 1), and wherein the transgene causes at least one pathological phenotypes associated with MPD1.

The novel MPD1 animal model of the invention may include the expression of the (3-myosin R1500P mutant transgene (SEQ ID NO. 1) that is under the control of a promoter, and more preferably a tissue-specific promoter. For example, the novel MPD1 animal model of the invention may include the expression of the β-myosin R1500P mutant transgene (SEQ ID NO. 1) that is further localized to the animal's fast skeletal muscle fibers. In this novel animal model, a nucleotide sequence encoding the β-myosin R1500P mutant transgene (SEQ ID NO. 1) may be operably linked with a tissue-specific promoter, and preferably an MCK promoter. This nucleotide sequence may be part of an expression vector, or other system to allow the introduction of the 3-myosin R1500P mutant transgene (SEQ ID NO. 1) to the subject animal to produce the animal model for the study of MPD1. In this embodiment, the animal model may include the stable integration and expression of the β-myosin R1500P mutant transgene (SEQ ID NO. 1) operably linked to an MCK promoter in the fast skeletal muscle fibers of a mouse, wherein the transgenic mouse exhibits at least one phenotype associated with MPD1. Additional embodiment of the novel MPD1 animal model may include a tag, such as a myc-tag coupled to the β-myosin R1500P mutant transgene (SEQ ID NO. 1) to allow the presence and activity of the protein in the transgenic animal to be better tracked and studied. As noted above, in this novel MPD1 animal model, the wild-type β-myosin may further be optionally knocked-out or its expression disrupted as generally describe elsewhere.

The invention may further include methods of screening or testing the efficacy of one or more potential therapeutic compounds, or other therapies directed to MPD1. In one preferred embodiment, a transgenic, non-human animal of the invention expressing the β-myosin R1500P mutant transgene (SEQ ID NO. 1) that exhibits at least one phenotype associated with Laing distal myopathy (MPD1) may be established. Next, and preferably a pharmaceutically effective amount of a therapeutic compound directed to the treatment of one or more pathological phenotypes associated MPD1 may be administered to the transgenic animal to determine if the therapeutic compound decreases one or more phenotypes associated with MPD1, and comparing any phenotype changes with an animal that did not receive the therapeutic compound or is not transgenic and expresses a wild-type β-myosin gene. Administration may be accomplished through a variety of routes, include oral, nasal, injection, and the like. Moreover, a therapeutic compound may include one or more small molecules, such as inhibitors of protein function or gene expression, or may include one or more biologic therapeutics, such as monoclonal or other antibody based treatments. Notably, a therapeutic compound may be part of a pharmaceutical composition, having a pharmaceutical carrier, which would be known by one of ordinary skill in the art.

As used herein, “pharmaceutically effective amount” means an amount of a therapeutic compound that is sufficient to significantly induce a physiological response, that preferable ameliorate one or more pathological phenotypes or symptoms of MPD1.

A therapeutic compound may be a small molecule, or other biologic composition that, when administered to a subject in need thereof, induces a physiological response, that preferably ameliorates one or more pathological phenotypes or symptoms of MPD1.

As noted above, the inventions includes a transgenic animal, and preferably a mouse, expressing the β-myosin R1500P mutant transgene that may produce one or more pathologies associated with MPD1. Notably, a transgenic animal can be prepared in a number of ways. A transgenic organism is one that has an extra or exogenous fragment of DNA incorporated into its genome, sometimes replacing an endogenous piece of DNA. At least for the purposes of this invention, any animal whose genome has been modified to introduce a R1500P β-myosin mutant transgene, as well as its mutant progeny, are considered transgenic animals. In order to achieve stable inheritance of the extra or exogenous DNA, the integration event must occur in a cell type that can give rise to functional germ cells. The two animal cell types that are used for generating transgenic animals are fertilized egg cells and embryonic stem cells. Embryonic stem (ES) cells can be returned from in vitro culture to a “host” embryo where they become incorporated into the developing animal and can give rise to transgenic cells in all tissues, including germ cells. The ES cells are transfected in culture and then the mutation is transmitted into the germline by injecting the cells into an embryo. The animals carrying mutated germ cells are then bred to produce transgenic offspring. The use of ES cells to make genetic changed in the mouse germline is well recognized. For a reviews of this technology, those of skill in the art are referred to Bronson and Smithies, J. Biol. Chem., 269(44), 27155-27158, (1994); Torres, Curr. Top. Dev. Biol., 36, 99-114; 1998 and the references contained therein.

Generally, blastocysts are isolated from pregnant mice at a given stage in development, for example, the blastocyst from mice may be isolated at day 4 of development (where day 1 is defined as the day of plug), into an appropriate buffer that will sustain the ES cells in an undifferentiated, pluripotent state. ES cell lines may be isolated by a number of methods well known to those of skill in the art. For example, the blastocysts may be allowed to attach to the culture dish and approximately 7 days later, the outgrowing inner cell mass picked, trypsinized and transferred to another culture dish in the same culture media. ES cell colonies appear 2-3 weeks later with between 5-7 individual colonies arising from each explanted inner cell mass. The ES cell lines can then be expanded for further analysis. Alternatively, ES cell lines can be isolated using the immunosurgery technique (described in Martin, Proc. Natl. Acad. Sci. USA 78:7634-7638 (1981)) where the trophectoderm cells are destroyed using anti-mouse antibodies prior to explanting the inner cell mass.

In generating transgenic animals, the ES cell lines that have been manipulated by homologous recombination are reintroduced into the embryonic environment by blastocyst injection (as described in Williams et al., Cell 52:121-131 (1988)). Briefly, blastocysts are isolated from a pregnant mouse and expanded. The expanded blastocysts are maintained in oil-drop cultures at 4° C. for 10 min prior to culture. The ES cells are prepared by picking individual colonies, which are then incubated in phosphate-buffered saline, 0.5 mM EGTA for 5 min; a single cell suspension is prepared by incubation in a trypsin-EDTA solution containing 1% (v/v) chick serum for a further 5 min at 4° C. Five to twenty ES cells (in Dulbecco's modified Eagle's Medium with 10% (v/v) fetal calf serum and 3,000 units/ml DNAase 1 buffered in 20 mM HEPES [pH 8]) are injected into each blastocyst. The blastocysts are then transferred into pseudo-pregnant recipients and allowed to develop normally. The transgenic mice are identified by coat markers (Hogan et al., Manipulating the Mouse Embryo, Cold Spring Harbor, N.Y. (1986)). Additional methods of isolating and propagating ES cells may be found in, for example, U.S. Pat. Nos. 5,166,065; 5,449,620; 5,453,357; 5,670,372; 5,753,506; 5,985,659, each incorporated herein by reference.

An alternative method involving zygote injection method for making transgenic animals is described in, for example, U.S. Pat. No. 4,736,866, incorporated herein by reference. Additional methods for producing transgenic animals are generally described by Wagner and Hoppe (U.S. Pat. No. 4,873,191; which is incorporated herein by reference), Brinster et al. Proc. Nat'l Acad. Sci. USA, 82(13) 4438-4442, 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

Briefly, this method involves injecting DNA into a fertilized egg, or zygote, and then allowing the egg to develop in a pseudo-pregnant mother. The zygote can be obtained using male and female animals of the same strain or from male and female animals of different strains. The transgenic animal that is born, the founder, is bred to produce more animals with the same DNA insertion. In this method of making transgenic animals, the new DNA typically randomly integrates into the genome by a non-homologous recombination event. One to many thousands of copies of the DNA may integrate at a site in the genome

Generally, the DNA is injected into one of the pronuclei, usually the larger male pronucleus. The zygotes are then either transferred the same day or cultured overnight to form 2-cell embryos and then transferred into the oviducts of pseudo-pregnant females. The animals born are screened for the presence of the desired integrated DNA.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 mg/ml in 5 mM Tris, pH 7.4 and 0.1 mM EDTA.

Additional methods for purification of DNA for microinjection are described in Hogan et al. Manipulating the Mouse Embryo (Cold Spring Harbor Laboratory, Cold Spring Harbor, N.Y., 1986), in Palmiter et al. Nature 300:611 (1982); in The Qiagenologist, Application Protocols, 3rd edition, published by Qiagen, Inc., Chatsworth, Calif.; and in Sambrook et al. Molecular Cloning: A Laboratory Manual (Cold Spring Harbor Laboratory, Cold Spring Harbor, N.Y., 1989).

In an exemplary microinjection procedure, female mice six weeks of age are induced to superovulate. The superovulating females are placed with males and allowed to mate. After approximately 21 hours, the mated females are sacrificed and embryos are recovered from excised oviducts and placed in an appropriate buffer, e.g., Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA in a 37.5° C. incubator with a humidified atmosphere at 5% CO₂, 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipette (about 10 to 12 embryos). The pipette tip is inserted into the infundibulum and the embryos transferred. After the transfer, the incision is closed by two sutures. The pregnant animals then give birth to the founder animals which are used to establish the transgenic line.

### Methods of Using Transgenic Cell and Animals

Transgenic animals and cell lines derived from such animals will find use in drug screening experiments. In this regard, heterozygotic R1500P β-myosin mutant transgenic animals and cell lines capable of expressing the mutant β-myosin gene will be exposed to test substances, to screen the test substances for the ability to decrease symptoms of depression, alter serotonin re-uptake or improve some other parameter normally associated with clinical depression. Therapeutic compounds identified by such procedures will be useful in the treatment of depression. Alternatively, the animals and cell lines may be useful for monitoring the effects of known antidepressants.

It is contemplated that this screening technique will prove useful in the general identification of a compound that will serve the purpose of overcoming, circumventing or otherwise abolishing the effects of wolframin deficit seen in wolframin heterozygotes. Such compounds may be useful in the treatment of various disorders related to wolframin syndrome as well as for the treatment of depression and depressive disorders.

In some embodiments of the various aspects described herein, a targeting vector can be used to introduce a modification of β-myosin, such as the R1500P β-myosin transgene. A “targeting vector” is a vector comprising sequences that can be inserted into the gene to be disrupted, e.g., by homologous recombination. The targeting vector generally has a 5′ flanking region and a 3′ flanking region homologous to segments of the gene of interest, surrounding a DNA sequence comprising a modification and/or a foreign DNA sequence to be inserted into the gene. For example, the foreign DNA sequence may encode a selectable marker, such as an antibiotics resistance gene Examples for suitable selectable markers are the neomycin resistance gene (NEO) and the hygromycin f-phosphotransferase gene. The 5′ flanking region and the 3′ flanking region are homologous to regions within the gene surrounding the portion of the gene to be replaced with the unrelated DNA sequence. In some embodiments, the targeting vector does not comprise a selectable marker. DNA comprising the targeting vector and the native gene of interest are contacted under conditions that favor homologous recombination. For example, the targeting vector can be used to transform embryonic stem (ES) cells, in which they can subsequently undergo homologous recombination.

A typical targeting vector contains nucleic acid fragments of not less than about 0.5 kb nor more than about 10.0 kb from both the 5′ and the 3′ ends of the genomic locus which encodes the gene to be modified (e.g. β-myosin). These two fragments are separated by an intervening fragment of nucleic acid which encodes the modification to be introduced. When the resulting construct recombines homologously with the chromosome at this locus, it results in the introduction of the modification, e.g. a deletion of an exon or the insertion of a stop codon.

The homologous recombination of the above-described targeting vectors is sometimes rare and such a construct can insert nonhomologously into a random region of the genome where it has no effect on the gene which has been targeted for deletion, and where it can potentially recombine so as to disrupt another gene which was otherwise not intended to be altered. In some embodiments, such non-homologous recombination events can be selected against by modifying the above-mentioned targeting vectors so that they are flanked by negative selectable markers at either end (particularly through the use of two allelic variants of tie thymidine kinase gene, the polypeptide product of which can be selected against in expressing cell lines in an appropriate tissue culture medium well known in the art—i.e. one containing a drug such as 5-bromodeoxyuridine). Non-homologous recombination between the resulting targeting vector comprising the negative selectable marker and the genome will usually result in the stable integration of one or both of these negative selectable marker genes and hence cells which have undergone non-homologous recombination can be selected against by growth in the appropriate selective media (e.g. media containing a drug such as 5-bromodeoxyuridine for example). Simultaneous selection for the positive selectable marker and against the negative selectable marker will result in a vast enrichment for clones in which the targeting vector has recombined homologously at the locus of the gene intended to be mutated.

In some embodiments, each targeting vector to be inserted into the cell is linearized. Linearization is accomplished by digesting the DNA with a suitable restriction endonuclease selected to cut only within the vector sequence and not the 5′ or 3′ homologous regions or the modification region. Thus, a targeting vector refers to a nucleic acid that can be used to decrease or suppress expression of a protein encoded by endogenous DNA sequences in a cell. In a simple example, the optional knockout construct is comprised of a β-myosin polynucleotide with a deletion in a critical portion of the polynucleotide (e.g the transmembrane domain) so that a functional β-myosin cannot be expressed therefrom. Alternatively, a number of termination codons can be added to the native polynucleotide to cause early termination of the protein or an intron junction can be inactivated. Proper homologous recombination can be confirmed by Southern blot analysis of restriction endonuclease digested DNA using, as a probe, a non-modified region of the gene. Since the native gene will exhibit a restriction pattern different from that of the disrupted gene, the presence of a disrupted gene can be determined from the size of the restriction fragments that hybridize to the probe.

A targeting vector can comprise the whole or a fragment of the genomic sequence of a β-myosin and optionally, a selection marker, e.g., a positive selection marker. Several kilobases of unaltered flanking DNA (both at the 5′ and 3′ ends) can be included in the vector (see e.g, Thomas and Capecchi, (1987) Cell, 51:503 for a description of homologous recombination vectors). In one aspect of the invention, the genomic sequence of the β-myosin gene comprises at least part of an exon of β-myosin, such as, such as the R1500P β-myosin transgene in one embodiment.

A selection marker of the invention can include a positive selection marker, a negative selection marker or include both a positive and negative selection marker. Examples of positive selection marker include but are not limited to, e.g., a neomycin resistance gene (neo), a hygromycin resistance gene, etc. In one embodiment, the positive selection marker is a neomycin resistance gene. In certain embodiments of the invention, the genomic sequence further comprises a negative selection marker. Examples of negative selection markers include but are not limited to, e.g., a diphtheria toxin gene, an HSV-thymidine kinase gene (HSV-TK), etc.

The term “modifier” is used herein to collectively refer to any molecule which can effect a modification of β-myosin, such as a knock-out of a wild-type β-myosin, or the transformation into the animal's genome of a R1500P β-myosin transgene, e.g. a targeting vector or a TALENs, CRISPR, or ZFN molecule, complex, and/or one or more nucleic acids encoding such a molecule or the parts of such a complex.

A modifier can be introduced into a cell by any technique that allows for the addition of the exogenous genetic material into nucleic genetic material can be utilized so long as it is not destructive to the cell, nuclear membrane, or other existing cellular or genetic structures. Such techniques include, but are not limited to transfection, scrape-loading or infection with a vector, pronuclear microinjection (U.S. Pat. Nos. 4,873,191, 4,736,866 and 4,870,009); retrovirus mediated transfer into germ lines (an der Putten, et al., Proc. Natl Acad. Sci., U.S 21, 82.6148-6152 (1985)); gene targeting in embryonic stem cells (Thompson, et al., Cell, 56:313-321 (1989)); nonspecific insertional inactivation using a gene trap vector (U.S. Pat. No. 6,436,707); electroporation of embryos (Lo, Mol. Cell Biol., 3:1803-1814 (1983)): lipofection and sperm-mediated gene transfer (Lavitrano, et al., Cell. 57:717-723 (1989)); each of which are incorporated by reference herein in its entirety. These methods and compositions can largely be broken down into two classes, viral based delivery systems and non-viral based delivery systems. For example, the modifier can be delivered through a number of direct delivery systems such as, electroporation, lipofection, calcium phosphate precipitation, plasmids, viral vectors, viral nucleic acids, phage nucleic acids, phages, cosmids, or via transfer of material in cells or carriers such as cationic liposomes. Appropriate means for transfection, including viral vectors, chemical transfectants, or physico-mechanical methods such as electroporation and direct diffusion of DNA, are described by, for example, Wolff J A., et al., Science, 247, 1465-1468, (1990); and Wolff, J A. Nature, 352, 815-818, (1991); each of which are incorporated by reference herein in its entirety. Such methods are well known in the art and readily adaptable for use with the compositions and methods described herein. The methods described herein can be used to deliver a modifier to any cell type, e.g. a germline cell, a zygote, an embryo, or a somatic cell. The cells can be cultured in vitro or present in vivo Non-limiting examples are provided herein below.

In one example, a modifier inserted into the nucleic genetic material by microinjection. Microinjection of cells and cellular structures is known and is used in the art. Following introduction of the transgene nucleotide sequence into the embryo, the embryo may be incubated in vitro for varying amounts of time, or reimplanted into the surrogate host, or both. In vitro incubation to maturity is within the scope of this invention One common method is to incubate the embryos in vitro for about 1-7 days, depending on the species, and then reimplant them into the surrogate host. In some embodiments, a zygote is microinjected. The use of zygotes as a target for modification of a host gene has an advantage in that in most cases the injected DNA will be incorporated into the host gene before the first cleavage (Brinster et al. (1985) PNAS 82:4438-4442). As a consequence, all cells of the transgenic animal will carry the incorporated nucleic acids of the targeting vector. This will in general also be reflected in the efficient transmission to offspring of the founder since 50% of the germ cells will harbor the modification. One route of introducing foreign DNA into a germ line entails the direct microinjection of linear DNA molecules into a pronucleus of a fertilized one-cell egg. Microinjected eggs are subsequently transferred into the oviducts of pseudopregnant foster mothers and allowed to develop. About 25% of the progeny inherit one or more copies of the micro-injected DNA Techniques suitable for obtaining transgenic animals have been amply described. A suitable technique for obtaining completely ES cell derived transgenic non-human animals is described in WO 98/06834.

In some embodiments, a modifier can be introduced into a cell by electroporation. The cells and the targeting vector can be exposed to an electric pulse using an electroporation machine and following the manufacturer's guidelines for use, After electroporation, the cells are typically allowed to recover under suitable incubation conditions. The cells are then screened for the presence of the targeting vector as explained herein.

Retroviral infection can also be used to introduce a nucleic acid modifier (e.g. a targeting vector) or a nucleic acid encoding a modifier into a cell, e.g. a non-human animal cell. In some embodiments, a retrovirus can be used to introduce the β-myosin modification, such as a R1500P β-myosin mutant transgene, to a cell or cells, e.g. an embryo. For example, the developing non-human embryo can be cultured in vitro to the blastocyst stage During this time, the blastomeres can be targets for retroviral infection (Jaenich, Proc. Natl. Acad. Sci. USA, 73:1260-1264 (1976)). Efficient infection of the blastomeres is obtained by enzymatic treatment to remove the zona pellucida (Manipulating the Mouse Embryo, Hogan, ed. (Cold Spring Harbor Laboratory Press, Cold Spring Harbor, N Y, 1986)). The viral vector system used to introduce the modifier is typically a replication-defective retrovirus carrying the transgene (Jahner et al., Proc. Natl. Acad. Sci. USA, 82: 6972-6931 (1985); and, Van der Putten et al., Proc. Natl. Acad. Sci. USA, 82: 6148-6152 (1985)) Transfection is easily and efficiently obtained by culturing the blastomeres on a monolayer of virus-producing cells (Van der Putten et al., supra; and, Stewart et al., EMBO J., 6: 383-388 (1987)). Alternatively, infection can be performed at a later stage. Virus or virus-producing cells can be injected into the blastocoele (Jahner et al., Nature, 298: 623-628(1982)). Most of the founders will be mosaic for the transgene since incorporation occurs only in a subset of the cells that formed the transgenic non-human animal. In addition, it is also possible to introduce transgenes into the germ line by intrauterine retroviral infection of the mid-gestation embryo (Jahner et al. (1982), supra).

Other viral vectors can include, but are not limited to, adenoviral vectors (Mitani et al., Hum Gene Ther 5:941-948, 1994), adeno-associated viral (AAV) vectors (Goodman et al., Blood 84:1492-1500, 1994), lentiviral vectors (Naidini et al., Science 272:263-267, 1996). pseudotyped retroviral vectors (Agrawal et al., Exper. Hematol. 24:738-747, 1996).

In some embodiments, a modifier can be introduced to a cell by the use of liposomes, e.g. cationic liposomes (e.g, DOTMA, DOPE, DC-cholesterol) or anionic liposomes. Liposomes can further comprise proteins to facilitate targeting a particular cell, if desired. Regarding liposomes, see, e.g., Brigham et al Am. J. Resp, Cell Mol. Biol. 1.95-100 (1989): Feigner et al. Proc. Natl. Acad. Sci. USA 84:7413-7417 (1987); U.S. Pat. No. 4,897,355; each of which is incorporated by reference herein in its entirety Commercially available liposome preparations include, e.g as LIPOFECTIN, LIPOFECIAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art.

The number of copies of a modifier (e.g., the targeting vector or TALENs molecule) which are added to the cell is dependent upon the total amount of exogenous genetic material added and will be the amount which enables the genetic transformation to occur, Theoretically only one copy is required; however, generally, numerous copies are utilized, for example, 1,000-20,000 copies of a targeting vector, in order to insure that one copy is functional.

In some embodiments, cells contacted with a modifier are subsequently screened for accurate targeting to identify and isolate those which have been properly modified at the β-myosin locus. Once the cell comprising a modification of β-myosin, such as a R1500P β-myosin mutant transgene, is produced through the methods described herein, an animal can be produced from this cell through either stem cell technology or cloning technology. For example, if the cell into which the nucleic acid was transfected was a stem cell for the organism (e.g. an embryonic stem cell), then this cell, after transfection and culturing, can be used to produce an organism which will contain the gene modification in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a β-myosin modification event take place, and then cells derived from this cell can be used to clone a whole animal.

Generally, cells (e.g. ES cells) used to produce the knockout animals will be of the same species as the knockout animal to be generated. Thus, for example, mouse embryonic stem cells will usually be used for generation of knockout mouse. Methods of isolating, culturing, and manipulating various cells types are known in the art. By way of non-limiting example, embryonic stem cells are generated and maintained using methods well known to the skilled artisan such as those described by Doetschman et al. (1985) J. Embryol. Exp. Mol. Biol. 87:27-45). The cells are cultured and prepared for knockout construct insertion using methods well known to the skilled artisan, such as those set forth by Robertson in: Teratocarcinomas and Embryonic Stem Cells: A Practical Approach, E. J. Robertson, ed. IRI. Press, Washington, D C. [1987]); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. (1986)).

In some embodiments, after cells comprising the modification of β-myosin have been generated, and optionally, selected, the cells can be inserted into an embryo. Insertion may be accomplished in a variety of ways known to the skilled artisan; however, the typical method is by microinjection. For microinjection, about 10-30 cells are collected into a micropipet and injected into embryos that are at the proper stage of development to permit integration of the ES cell containing the β-myosin modification, such as the R1500P β-myosin transgene, into the developing embryo. For instance, the ES cells can be microinjected into blastocytes. The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan.

In some embodiments, a modification of β-myosin that renders it nonfunctional can be generated by a recombinase. For example, sites for a recombinase can be inserted into the native β-myosin gene, such that they flank an area that can be deleted in order to render β-myosin nonfunctional (e.g. exon 3). In the presence of the recombinase, the flanked area of β-myosin will be deleted. This permits inducible or tissue-specific modification of β-myosin, e.g. in the brain only.

A widely used site-specific DNA recombination system uses the Cre recombinase, e.g., from bacteriophage P1, or the Flp recombinase from S5 cerevisiae, which has also been adapted for use in animals. The loxP-Cre system utilizes the expression of the PI phage Cre recombinase to catalyze the excision of DNA located between flanking lox sites. By using gene-targeting techniques to produce binary transgene animals with modified endogenous genes that can be acted on by Cre or Flp recombinases expressed under the control of tissue-specific promoters, site-specific recombination may be employed to inactivate endogenous genes in a spatially or time controlled manner. See, e.g., U.S. Pat. Nos. 6,080,576, 5,434,066, and 4,959,317, and Joyner, A L., et al. Laboratory Protocols for Conditional Gene Targeting, Oxford University Press, New York (1997). The cre-lox system, an approach based on the ability of transgenic mice, carrying the bacteriophage Cre gene, to promote recombination between, for example, 34 by repeats termed loxP sites, allows ablation of a given gene in a tissue specific and a developmentally regulated manner (Orban et al. (1992) PNAS 89:6861-6865). The Cre-lox system has been successfully applied for tissue-specific transgene expression (Orban P C, Chui D, Marth J D. Proc Natl Acad Sci USA. 1992 Aug. 1; 89(15) 6861-5.), for site specific gene targeting and for exchange of gene sequence by the “knock-in” method (Aguzzi A Brandner S, Isenmann S. Steinbach J P, Sure U. Glia. 1995 November, 15(3):348-64. Review).

The recombinase can be delivered at different stages. For example, a recombinase can be added to an embryonic stem cell containing a disrupted gene prior to the production of chimeras or implantation into an animal. In certain embodiments of the invention, the recombinase is delivered after the generation of an animal containing at least one gene allele with introduced recombinase sites. For example, the recombinase is delivered by cross breeding the animal containing a gene with recombinase sites with an animal expressing the recombinase. The animal expressing the recombinase may express it, e.g., ubiquitously, in a tissue-restricted manner, or in a temporal-restricted manner. Cre/Flp activity can also be controlled temporally by delivering cre/FLP-encoding transgenes in viral vectors, by administering exogenous steroids to the animals that carry a chimeric transgene consisting of the cre gene fused to a mutated ligand-binding domain, or by using transcriptional transactivation to control cre/FLP expression. In certain embodiments of the invention, mutated recombinase sites may be used. Tissue-specific, temporally-regulated, and inducible promoters for controlling the expression of, e.g. Cre recombinase are known in the art.

Animals with germline cells comprising the desired modification can be selected, e.g. by genotyping or assaying s-myosin levels or activity in the germline cells and/or progeny. Non-limiting examples of methods for such genotyping or assaying can include, RNA analysis (Northern blotting or RT-PCR, including qRT-PCR), assays for determining the activity of pi-myosin as described elsewhere herein, protein analysis (e.g. Western blotting), histological stains, flow cytometric analysis and the like. The extent of the contribution of the modified cells in an animal described herein can also be assessed visually by choosing animals strains for the modified cells (e.g. the ES cells that will be modified) and the blastocytes that have different coat colors. Transgenic offspring can be screened for the presence and/or expression of the transgene by any suitable method. Screening is often accomplished by Southern blot or Northern blot analysis, using a probe that is complementary to at least a portion of the transgene. Typically, DNA is prepared from, e.g. tail tissue and analyzed by Southern analysis or PCR for the transgene. Alternatively, the tissues or cells believed to have a modification of β-myosin are tested for the presence and expression of the modified β-myosin using Southern analysis or PCR, although any tissues or cell types may be used for this analysis. See, e.g., southern hybridization. (Southern J. Mol. Biol. 98:503-517 (1975)), northern hybridization (see, e.g., Freeman et al Proc. Natl Acad. Sci. USA 80:4094-4098 (1983)), restriction endonuclease mapping (Sambrook et al. (2001) Molecular Cloning, A Laboratory Manual, 3rd Ed. Cold Spring Harbor Laboratory Press, New York), RNase protection assays (Current Protocols in Molecular Biology, John Wiley and Sons, New York, 1997), DNA sequence analysis, and polymerase chain reaction amplification (PCR, U.S. Pat. Nos. 4,683,202, 4,683,195, and 4,889,818; Gyllenstein et al. Proc Natl. Acad. Sci. USA 85:7652-7657 (1988); Ochman et al. Genetics 120:621-623 (1988); and, Loh et al. Science 243:217-220 (1989). Other methods of amplification commonly known in the art can be employed. The stringency of the hybridization conditions for northern or Southern blot analysis can be manipulated to ensure detection of nucleic acids with the desired degree of relatedness to the specific probes used. The expression of gene in a cell or tissue sample can also be detected and quantified using in situ hybridization techniques according to, for example, Current Protocols in Molecular Biology, John Wiley and Sons, New York, 1997.

Protein levels can be detected by immunoassays using antibodies specific to the protein. For example, western blot analysis using an antibody against s-myosin or the modified β-myosin encoded by the transgene may be employed as an alternative or additional method for screening for the presence of the transgene product. Various immunoassays known in the art can be used, including but not limited to competitive and non-competitive assay systems using techniques such as radioimmunoassay, ELISA (enzyme linked immunosorbant assay), “sandwich” immunoassays, immunoradiometric assays, gel diffusion precipitin reactions, immunodiffusion assays, in situ immunoassays (using colloidal gold, enzyme or radioisotope labels), western blot analysis, precipitation reactions, agglutination assays (e.g., gel agglutination assays, hemagglutination assays), complement fixation assays, immunofluorescence assays, protein A assays, immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or optionally a knockout; where it is transgenic, it may contain the same or a different knockout, or both. Alternatively, the partner may be a parental line. Where in vitro fertilization is used, the fertilized embryo may be implanted into a surrogate host or incubated in vitro, or both. Using either method, the progeny may be evaluated using methods described above, or other appropriate methods.

In some embodiments a cell and/or animal described herein can comprise one or more additional modifications or transgenes (e.g. an additional transgene and/or second knockout targeting a gene other than β-myosin). Cells and/or animals containing more than one knockout construct and/or more than one transgene expression construct can be prepared in any of several ways. A typical manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

Described herein are nucleic acid molecules comprising a modification of β-myosin, such as the R1500P β-myosin transgene, as described herein, e.g. a targeting vector comprising a modified variant of β-myosin, such as the R1500P β-myosin transgene, according to the embodiments described herein. Described herein are cells produced by the process of transforming the cell with any of the described nucleic acids. Described herein are cells produced by the process of transforming the cell with any of the non-naturally occurring described nucleic acids. Described herein are peptides produced by the process of expressing any of the described nucleic acids. Described herein are any of the non-naturally occurring peptides produced by the process of expressing any of the described nucleic acids. Described herein are any of the described peptides produced by the process of expressing any of the non-naturally occurring described nucleic acids. Described herein are animals produced by the process of transfecting a cell within the animal with any of the nucleic acid molecules described herein. Described herein are animals produced by the process of transfecting a cell within the animal any of the nucleic acid molecules described herein, wherein the animal is a mammal. Described herein are animals produced by the process of adding to the animal any of the cells described herein.

As used herein, “knock-out” refers to partial or complete suppression of the expression of a protein encoded by an endogenous DNA sequence in a cell. The “knock-out” can be affected by targeted deletion of the whole or part of a gene encoding a protein in a cell. In some embodiments, the deletion may prevent or reduce the expression of the functional protein in any cell in the whole, or part of the animal in which it is normally expressed. For example, a “β-myosin knock-out animal” refers to an animal in which the expression of functional β-myosin has been reduced or suppressed by the introduction of a recombinant modifier that introduces a modification in the sequence of the β-myosin gene. A knock-out animal can be a transgenic animal, or can be created without transgenic methods. e.g. by transient introduction of a TALENs molecule, such that a deletion of part or all of the β-myosin gene occurs, but without the introduction of exogenous DNA to the genome.

In certain embodiments, a transgenic animal or cell-line of the invention may be created using gene-editing endonucleases such as CRISPR/Cas9, Zinc-fingers, and TALENS. For example, Zinc finger nucleases (ZFNs), the Cas9/CRISPR system, and transcription-activator like effector nucleases (TALENs) are meganucleases. Meganucleases are found commonly in microbial species and have the unique property of having very long recognition sequences (>14 bp) thus making them naturally very specific for cutting at a desired location. This can be exploited to make site-specific double-stranded breaks in, e.g. a genome. These nucleases can cut and create specific double-stranded breaks at a desired location(s) in the genome, which are then repaired by cellular endogenous processes such as, homologous recombination (HR), homology directed repair (HDR) and non-homologous end-joining (NHEJ). NHEJ directly joins the DNA ends in a double-stranded break, while HDR utilizes a homologous sequence as a template for regenerating the missing DNA sequence at the break point. Thus, by introducing a ZFN, CRISPR, and/or TALENs specific for β-myosin into a cell, at least one double strand-break can be generated in $i-myosin, resulting in an excision of at least part of the β-myosin gene (i.e. introducing a modification as described herein) (see, e.g. Gaj et al. Trends in Biotechnology 2013 31:397-405; Carlson et al. PNAS 2012 109:17382-7, and Wang et al. Cell 2013 153.910-8; each of which is incorporated by reference herein in its entirety). Alternatively, if a specifically-designed homologous donor DNA is provided in combination with, e.g., the ZFNs, this template can result in gene correction or insertion, as repair of the DSB can include a few nucleotides changed at the endogenous site or the addition of a new and/or modified gene at the break site. One of skill in the art can use these naturally occurring meganucleases, however the number of such naturally occurring meganucleases is limited.

In some embodiments, the Cas9/CRISPR system can be used to create a modification, such as a knock-out, in an β-myosin gene as described herein. Clustered regularly interspaced short palindromic repeats (CRISPR)/CRISPR-associated (Cas) systems are useful for, e.g RNA-programmable genome editing (see e.g., Marraffini and Sontheimer. Nature Reviews Genetics 2010 11:18-190, Sorek et al. Nature Reviews Microbiology 2008 6:181-6; Karginov and Hannon. Mol Cell 2010 1:7-19; Hale et al. Mol Cell 2010:45:292-302: Jinek et al. Science 2012 337:815-820: Bikard and Marraffini Curr Opin Immunol 2012 24:15-20: Bikard et al. Cell Host & Microbe 2012 12:177-186; all of which are incorporated by reference herein in their entireties). A CRISPR guide RNA is used that can target a Cas enzyme to the desired location in the genome, where it generates a double strand break. This technique is known in the art and described, e.g. at Mali et al. Science 2013 339:823-6; which is incorporated by reference herein in its entirety and kits for the design and use of CRISPR-mediated genome editing are commercially available, e.g. the PRECISION X CAS9 SMART NUCLEASE™ System (Cat No. CAS900A-1) from System Biosciences, Mountain View, Calif. More generally, as used herein, CRISPR/Cas9 technology generally encompasses an RNA-guided gene-editing platform that makes use of a bacterially derived protein (Cas9) and a synthetic gRNA to introduce a double-strand break at a specific location within the genome of the eukaryotic host Generally, CRISPR/Cas9 may be used to generate a knock-out or disrupt or replace target gene, such a β-myosin gene by co-expressing a gRNA specific to the gene to be targeted and the endonuclease Cas9. CRISPR may consist of two components: gRNA and a non-specific CRISPR-associated endonuclease (Cas9). The gRNA may be a short synthetic RNA composed of a scaffold sequence that may allow for Cas9-binding and a ˜20 nucleotide spacer or targeting sequence which defines the genomic target to be modified.

The term “zinc finger,” as used herein, refers to a small nucleic acid-binding protein structural motif characterized by a fold and the coordination of one or more zinc ions that stabilize the fold Zinc fingers encompass a wide variety of differing protein structures (see, e.g, Klug A, Rhodes D (1987). “Zinc fingers: a novel protein fold for nucleic acid recognition”. Cold Spring Harb. Symp Quant Biol. 52: 473-82, the entire contents of which are incorporated herein by reference). Zinc fingers can be designed to bind a specific sequence of nucleotides, and zinc finger arrays comprising fusions of a series of zinc fingers, can be designed to bind virtually any desired target sequence Such zinc finger arrays can form a binding domain of a protein, for example, of a nuclease, e.g., if conjugated to a nucleic acid cleavage domain. Different types of zinc finger motifs are known to those of skill in the art, including, but not limited to, Cys2His2, Gag knuckle, Treble clef, Zinc ribbon, Zn2/Cys6, and TAZ2 domain-like motifs (see, e.g., Krishna S S, Majumdar I, Grishin N V (January 2003). “Structural classification of zinc fingers: survey and summary”. Nucleic Acids Res. 31 (2): 532-50). Typically, a single zinc finger motif binds 3 or 4 nucleotides of a nucleic acid molecule. Accordingly, a zinc finger domain comprising 2 zinc finger motifs may bind 6-8 nucleotides, a zinc finger domain comprising 3 zinc finger motifs may bind 9-12 nucleotides, a zinc finger domain comprising 4 zinc finger motifs may bind 12-16 nucleotides, and so forth. Any suitable protein engineering technique can be employed to alter the DNA-binding specificity of zinc fingers and/or design novel zinc finger fusions to bind virtually any desired target sequence from 3-30 nucleotides in length (see, e.g, Pabo C O, Peisach E, Grant R A (2001). “Design and selection of novel cys2H is2 Zinc finger proteins”. Annual Review of Biochemistry 70: 313-340, Jamieson A C, Miller J C, Pabo C O (2003) “Drug discovery with engineered zinc-finger proteins”. Nature Reviews Drug Discovery 2 (5): 361-368; and Liu Q, Segal D J, Ghiara J B, Barbas C F (May 1997). “Design of poly dactyl zinc-finger proteins for unique addressing within complex genomes”. Proc. Natl. Acad. Sci. U.S.A. 94 (11); the entire contents of each of which are incorporated herein by reference).

Fusions between engineered zinc finger arrays and protein domains that cleave a nucleic acid can be used to generate a “zinc finger nuclease.” A zinc finger nuclease typically comprises a zinc finger domain that binds a specific target site within a nucleic acid molecule and a nucleic acid cleavage domain that cuts the nucleic acid molecule within or in proximity to the target site bound by the binding domain. Typical engineered zinc finger nucleases comprise a binding domain having between 3 and 6 individual zinc finger motifs and binding target sites ranging from 9 base pairs to 18 base pairs in length Longer target sites are particularly attractive in situations where it is desired to bind and cleave a target site that is unique in a given genome. Zinc finger nucleases can be generated to target a site of interest by methods well known to those of skill in the art. For example, zinc finger binding domains with a desired specificity can be designed by combining individual zinc finger motifs of known specificity. The structure of the zinc finger protein Zif268 bound to DNA has informed much of the work in this field and the concept of obtaining zinc fingers for each of the 64 possible base pair triplets and then mixing and matching these modular zinc fingers to design proteins with any desired sequence specificity has been described (Pavletich N P, Pabo Colo. (May 1991). “Zinc fmger-DNA recognition: crystal structure of a Zif268-DNA complex at 2.1 A” Science 252 (5007) 809-17, the entire contents of which are incorporated herein). In some embodiments, separate zinc fingers may be generated that each recognizes a 3 base pair DNA sequence are combined to generate 3-, 4-, 5-, or 6-finger arrays that recognize target sites ranging from 9 base pairs to 18 base pairs in length. In some embodiments, longer arrays are contemplated. In other embodiments, 2-finger modules recognizing 6-8 nucleotides are combined to generate 4-, 6-, or 8-zinc finger arrays. In some embodiments, bacterial or phage display is employed to develop a zinc finger domain that recognizes a desired nucleic acid sequence, for example, a desired nuclease target site of 3-30 bp in length.

As noted above, zinc finger nucleases, in some embodiments may comprise a zinc finger binding domain and a cleavage domain fused or otherwise conjugated to each other via a linker, for example, a polypeptide spacer. The length of the linker determines the distance of the cut from the nucleic acid sequence bound by the zinc finger domain. If a shorter linker is used, the cleavage domain will cut the nucleic acid closer to the bound nucleic acid sequence, while a longer linker will result in a greater distance between the cut and the bound nucleic acid sequence. In some embodiments, the cleavage domain of a zinc finger nuclease has to dimerize in order to cut a bound nucleic acid. In some such embodiments, the dimer is a heterodimer of two monomers, each of which comprise a different zinc finger binding domain. For example, in some embodiments, the dimer may comprise one monomer comprising zinc finger domain A conjugated to a Fokl cleavage domain, and one monomer comprising zinc finger domain 13 conjugated to a Fokl cleavage domain. In this non-limiting example, zinc finger domain A binds a nucleic acid sequence on one side of the target site, zinc finger domain B binds a nucleic acid sequence on the other side of the target site, and the dimerize Fokl domain cuts the nucleic acid in between the zinc finger domain binding sites.

The term TALEN or “Transcriptional Activator-Like Element Nuclease” or “TALE nuclease” as used herein, refers to an artificial nuclease comprising a transcriptional activator like effector DNA binding domain to a DNA cleavage domain, for example, a Fokl domain. A number of modular assembly schemes for generating engineered TALE constructs have been reported (Zhang, Feng; et. al. (February 2011). “Efficient construction of sequence-specific TAL effectors for modulating mammalian transcription” Nature Biotechnology 29 (2): 149-53, Geibler, R, Scholze, H.: Hahn, S.; Streubel, J.; Bonas, U.; Behrens, S. E.; Boch, J. (2011). Shiu, Shin-Han. ed. “Transcriptional Activators of Human Genes with Programmable DNA-Specificity”. PLoS ONE 6 (5): e19509: Cermak, T.; Doyle, E. L.: Christian, M.; Wang, L.; Zhang, Y.; Schmidt, C.; Baller, J. A.; Somia, N. V et al. (2011) “Efficient design and assembly of custom TALEN and other TAL effector-based constructs for DNA targeting”. Nucleic Acids Research: Morbitzer, R.; Elsaesser, J.; Hausner, J, Lahaye, T. (2011). “Assembly of custom TALE-type DNA binding domains by modular cloning”. Nucleic Acids Research; Li, T.; Huang, S.; Zhao, X.; Wright, D. A.: Carpenter, S.; Spalding, M. H.; Weeks, D. P.; Yang, B. (2011). “Modularly assembled designer TAL effector nucleases for targeted gene knockout and gene replacement in eukaryotes”. Nucleic Acids Research.; Weber, E.; Gruetzner, R.; Werner, S.; Engler, C.; Marillonnet, S. (2011). Bendahmane, Mohammed ed “Assembly of Designer TAL Effectors by Golden Gate Cloning” PLoS ONE 6 (5): e19722; each of which is incorporated herein by reference). Those of skill in the art will understand that TALE nucleases can be engineered to target virtually any genomic sequence with high specificity, and that such engineered nucleases can be used in embodiments of the present technology to manipulate the genome of a cell, e.g., by delivering the respective TALEN via a method or strategy disclosed herein under circumstances suitable for the TALEN to bind and cleave its target sequence within the genome of the cell. In some embodiments, the delivered TALEN targets a gene or allele associated with a disease or disorder or a biological process, or one or more target genes.

As used herein, a “transgenic animal” or “genetically modified” animal refers to an animal, and preferably a mouse, to which exogenous DNA has been introduced. In most cases, the transgenic approach aims at specific modifications of the genome, e.g., by introducing whole transcriptional units into the genome, or by up- or down-regulating pre-existing cellular genes. The targeted character of certain of these procedures sets transgenic technologies apart from experimental methods in which random mutations are conferred to the germline, such as administration of chemical mutagens or treatment with ionizing solution.

The term “animal” is used herein to include all vertebrate animals, except humans. It also includes an individual animal in all stages of development, including embryonic, fetal stages and germ cell lines. For example, a germ cell line of a transgenic animal refers to a transgenic animal in which the genetic alteration or genetic information was introduced into a germ line cell, thereby conferring the ability to transfer the genetic information to offspring. If such offspring in fact possess some or all of that alteration or genetic information, they are transgenic animals as well. The term “chimera,” “mosaic,” “chimeric animal” and the like, refers to a transgenic and/or knock-out animal with exogenous DNA and/or a modification of β-myosin in some of its genome-containing cells.

The term “heterozygote,” “heterozygotic” and the like, refers to a transgenic and/or knock-out animal with exogenous DNA and/or a modification of the β-myosin gene, such as the R1500P mutation of the invention, on one of a chromosome pair in all of its genome-containing cells. The term “homozygote,” “homozygotic” and the like, refers to a transgenic mammal with exogenous DNA and/or a modification of the β-myosin gene, such as the R1500P mutation of the invention, on both members of a chromosome pair in all of its genome-containing cells.

The term “homologous recombination” refers to the exchange of DNA fragments between two DNA molecules or chromatids at the site of homologous nucleotide sequences. The term “gene targeting” refers to a type of homologous recombination that occurs when a fragment of genomic DNA is introduced into a mammalian cell and that fragment locates and recombines with endogenous homologous sequences. Gene targeting by homologous recombination employs recombinant DNA technologies to replace specific genomic sequences with exogenous DNA of particular design and/or a modified sequence.

The term “wild type” or “wild type expression” refers to the expression of the full-length polypeptide encoded by a gene, e.g., a β-myosin gene, at expression levels present in the wild-type cell and/or animal.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 1-10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more up to 100%.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA. Notably, where a nucleotide sequence is provided, the corresponding amino acid sequence is also encompassed within the disclosure and definition. Conversely, where an amino acid sequence is provided, the corresponding nucleotide sequence is also encompassed within the disclosure and definition.

An “isolated” nucleic acid molecule is a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells.

The term “gene” refers to (a) a gene containing a DNA sequence encoding a protein, e.g, β-myosin or mutant R1500P β-myosin transgene; (b) any DNA sequence that encodes a protein, e.g., or mutant R1500P β-myosin transgene gene amino acid sequence, and/or; (c) any DNA sequence that hybridizes to the complement of the coding sequences of a protein. In certain embodiments, the term includes coding as well as noncoding regions, and preferably includes all sequences necessary for normal gene expression.

As used herein, the term “genome” refers to chromosomal DNA found within the nucleus of a cell, and also refers to organelle DNA found within subcellular components of the cell.

The term, “operably linked,” when used in reference to a regulatory sequence and a coding sequence, means that the regulatory sequence affects the expression of the linked coding sequence. “Regulatory sequences,” or “control elements,” refer to nucleotide sequences that facilitate the transcription of eukaryotic-like mRNAs in prokaryotic cells, and/or facilitate the export of eukaryotic-like mRNAs out of a prokaryotic cells, and/or facilitate the uptake of eukaryotic-like mRNAs by eukaryotic cells, and/or facilitate the translation of eukaryotic-like mRNAs in eukaryotic cells. The terms may additionally encompass nucleotide sequences that influence the timing and level/amount of transcription, RNA processing or stability, or translation of the associated coding sequence. Regulatory sequences may include promoters, translation leader sequences: introns; enhancers; stem-loop structures; repressor binding sequences: termination sequences; polyadenylation recognition sequences and the like. Particular regulatory sequences may be located upstream and/or downstream of a coding sequence operably linked thereto. Also, particular regulatory sequences operably linked to a coding sequence may be located on the associated complementary strand of a double-stranded nucleic acid molecule. As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. A“plant promoter” may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters which initiate transcription only in certain tissues are referred to as “tissue-specific.”

A “cell type-specific” promoter primarily drives expression in certain cell types in one or more organs, for example, vascular cells in roots or leaves. An“inducible” promoter may be a promoter which may be under environmental control. Examples of environmental conditions that may initiate transcription by inducible promoters include anaerobic conditions and the presence of light Tissue-specific, tissue-preferred, cell type specific, and inducible promoters constitute the class of “non-constitutive” promoters. A “constitutive” promoter is a promoter which may be active under most environmental conditions or in most cell or tissue types.

An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s).

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et ah, Nucleic Acid Res. 19:5081 (1991); Ohtsuka et ah, J. Biol. Chem. 260:2605-2608 (1985); and Rossolini et ah, Mol. Cell. Probes 8.91-98 (1994)) Because of the degeneracy of nucleic acid codons, one can use various different polynucleotides to encode identical polypeptides.

The terms “increase”, “increased”, “upregulate”, or “upregulation” are all used herein to mean an increase by a statistically significant amount. In some embodiments, “increase”, “increased”, “upregulate”, or “upregulation” typically means an increase by at least 1-10% as compared to a reference level (e.g. a control) and can include, for example, an increase by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more up to 100%, or an any increasing multiple after that.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.”

Definitions of common terms in cell biology and molecular biology can be found in The Encyclopedia of Molecular Biology, published by Blackwell Science Ltd, 1994 (ISBN 0-632-02182-9: Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds. Unless otherwise stated, the present invention was performed using standard procedures, as described, for example in Sambrook et al., Molecular Cloning: A Laboratory Manual (3 ed), Cold Spring Harbor Laboratory Press, Cold Spring Harbor, N.Y., USA (2001); Davis et al., Basic Methods in Molecular Biology, Elsevier Science Publishing, Inc., New York, USA (1995), Current Protocols in Protein Science (CPPS) (John E. Coligan, et al., ed, John Wiley and Sons, Inc.), Current Protocols in Cell Biology (CPCB) (Juan S. Bonifacino et. al. ed., John Wiley and Sons, Inc.), and Culture of Animal Cells. A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1 st edition, 1998) which are all incorporated by reference herein in their entireties.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1. Generation and Characterization of R1500P Mice

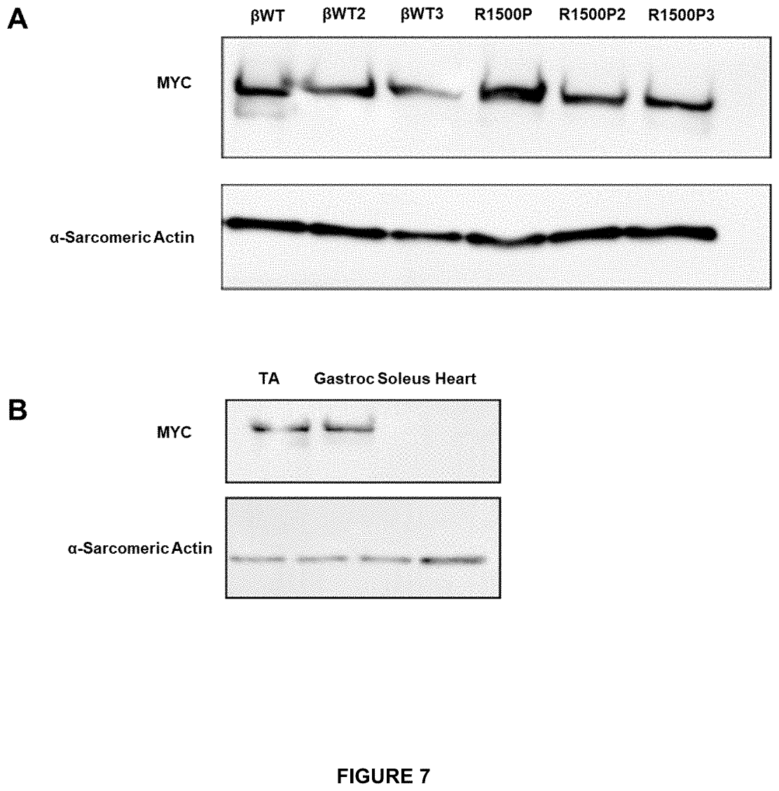

To create a mouse model for MPD1, the present inventors generated mice expressing the β-myosin R1500P mutation under the control of the well-characterized muscle creatine kinase (MCK) promoter that restricts transgene expression to fast skeletal muscle fibers only. This strategy was followed to circumvent the much lower abundance of slow/Type I fibers in the mouse compared to human (FIG. 1A), and therefore increase the opportunity to reveal a muscle phenotype. Expression of the transgene was tracked by tagging the C-terminal end of the mouse rod domain, which is ˜99% identical to the human homolog (SEQ ID NO. 3), with a myc-tag epitope. As a control, mice expressing myc-tagged WT 3-MyHC were also created (FIG. 1A). Three transgenic lines were created for each group, all expressing similar amounts of WT and mutant transgene protein (FIG. 7A). Moreover, the present inventor found that in tibialis anterior (TA) muscle, their expression levels are ˜35% of total myosin. (FIG. 1). In agreement with the MCK promoter specificity, transgene expression was not detected in any muscles that normally express detectable amounts of Type I/slow myosin such as soleus and heart (FIG. 7B).

### Example 2. Characteristic MPD1 Histopathology is Present in R1500P Muscles

Histological features associated with MPD1 are frequently variable and can include: i) change in muscle fiber size with type I hypotrophy, ii) co-expression of slow and fast myosins, iii) core/minicore structures, iv) mitochondrial abnormalities, and v) muscle necrosis and regeneration. Hence, the present inventors next determined whether the expression of the R1500P mutant in the mouse model induces some of the pathological phenotypes observed in human muscles. The present inventors found that while expression of the R1500P mutant did not change the whole muscle weight of measured fast-type muscles, it significantly decreased the muscle/body weight ratio when compared to the βWT transgenic control (FIG. 1C). Histological analysis of WT and R1500P TA muscle enzymatically stained for the mitochondrial enzyme succinate dehydrogenase (SDH) showed no significant difference in the percentage of positive fibers (FIG. 1D).

However, while the proportion of fast versus slow muscle fibers was unchanged, measurement of fiber cross-sectional area showed that R1500P muscle had a higher proportion of smaller muscle fibers than the βWT control (FIG. 1D). Thus, fiber hypotrophy could account for the observed decrease in muscle/body weight. To measure the level of expression of the different myosin heavy chain isoforms in TA muscle, the present inventors carried out quantitative real-time PCR (qRT-PCR). It was found that RNA for both the slowest and fastest myosin isoforms (Myh7 and Myh4 respectively) were upregulated by R1500P mutant expression, while the RNA level of the intermediate skeletal myosin isoforms Myh2 and Myh1 were not affected (FIG. 1E).

However, while Myh4 RNA was upregulated, the same was not observed at the protein level. To determine if expression of the R1500P mutant myosin led to sarcomere disorganization, the present inventors next complemented these studies by analyzing TA muscle ultrastructure with transmission electron microscopy (TEM). This analysis revealed that the integrity of the major sarcomeric components was not affected. However, ultrastructural changes in the sarcoplasmic reticulum (SR), t-tubules, and mitochondria were observed in the R1500P animals. While WT muscles showed the normal pattern of tightly wound SR networks with accompanying t-tubules triads, R1500P muscles had distended, irregular, and enlarged SR with the t-tubules having a variety of abnormalities ranging from mild to severe dilation of the triad structure (FIG. 2A). Furthermore, as confirmed by quantification of mitochondrial DNA content, the number of mitochondria was also decreased (FIG. 2B). Taken together, these results demonstrate that a number of the histological hallmarks that are often identified in patients with MPD1 are also present in our transgenic mouse model. Moreover, they provide evidence that the SR structure and/or function could also be altered by the expression of the R1500P mutant myosin.

### Example 3. The Presence of the R1500P Mutation Activates Genes Involved in Skeletal Muscle ER Stress and the Unfolded Protein Response (UPR)

Changes and/or disruptions to the ultrastructure of skeletal muscle can cause endoplasmic reticulum (ER) stress, which affects proper ER function by increasing the amount of misfolded/unfolded proteins in the ER lumen. As a result, a homeostatic signaling pathway, called Unfolded Protein Response (UPR) is activated. UPR inhibits protein synthesis, increases ER concentration of chaperones, and ultimately, triggers apoptosis. Since recent evidence has indicated that UPR is upregulated in a variety of myopathies, the present inventors measured the RNA levels of members of the PERK pathway, which is one of the major UPR transducers and has previously been shown to be activated in muscular dystrophy. In TA muscle of R1500P mice, significant upregulation of PERK and downstream members of the pathway including ATF4, ATF3, and GADD34 were identified (FIG. 3A-D). Other related members of the PERK pathway, such as CHOP were, however, unaffected by the presence of the R1500P mutation (FIG. 3E). Thus, in skeletal muscle, heightened activation of genes involved in the UPR pathways may contribute to the development of the MPD1 phenotype.

### Example 4. Muscle Fitness and Strength are Decreased in R1500P Mice

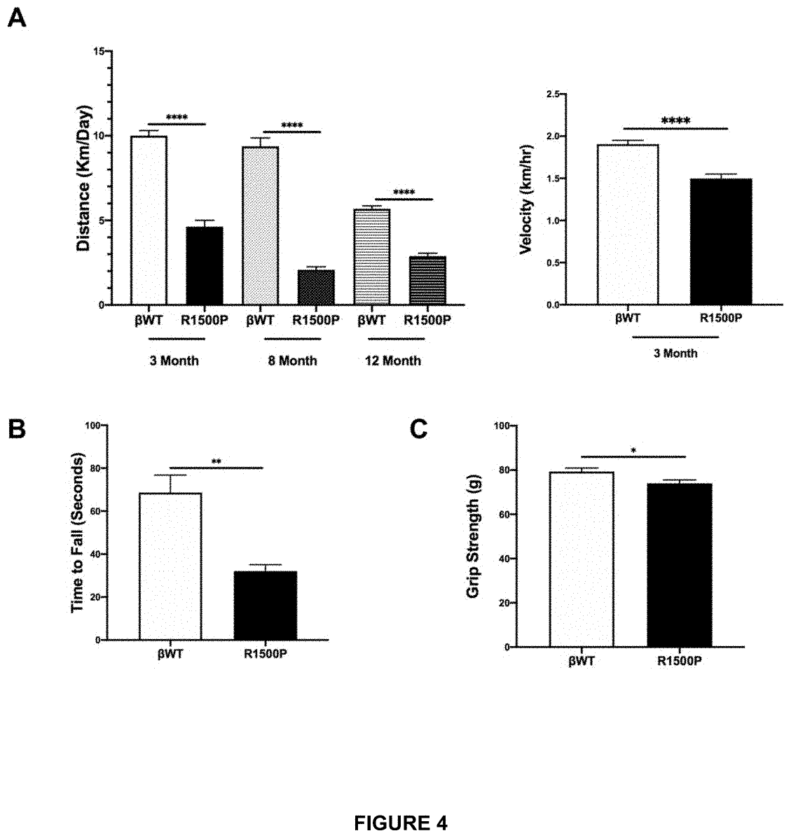

To determine whether the R1500P mutation hinders the biochemical mechanics of muscle contraction, the present inventors first measured exercise tolerance and fitness in βWT and R1500P mice using a fully automated tracking system to monitor voluntary wheel running. This analysis showed that both the running speed and total running distance over a 28-day period was significantly reduced in 3, 8- and 12-month-old male R1500P mice when compared to their βWT counterparts (FIG. 4A). Consistent with these results, muscle strength analyses including a four-limb hanging test and grip strength measurements were impaired with decreased average hang time and a significant reduction in force output in R1500P animals (FIG. 4B, 4C). Thus, as seen in MPD1 patients, the expression of the R1500P mutant in the mouse model affects muscle performance and strength.

### Example 5. Ex-Vivo Contractility Assay Shows Altered R1500P Muscle Contractility

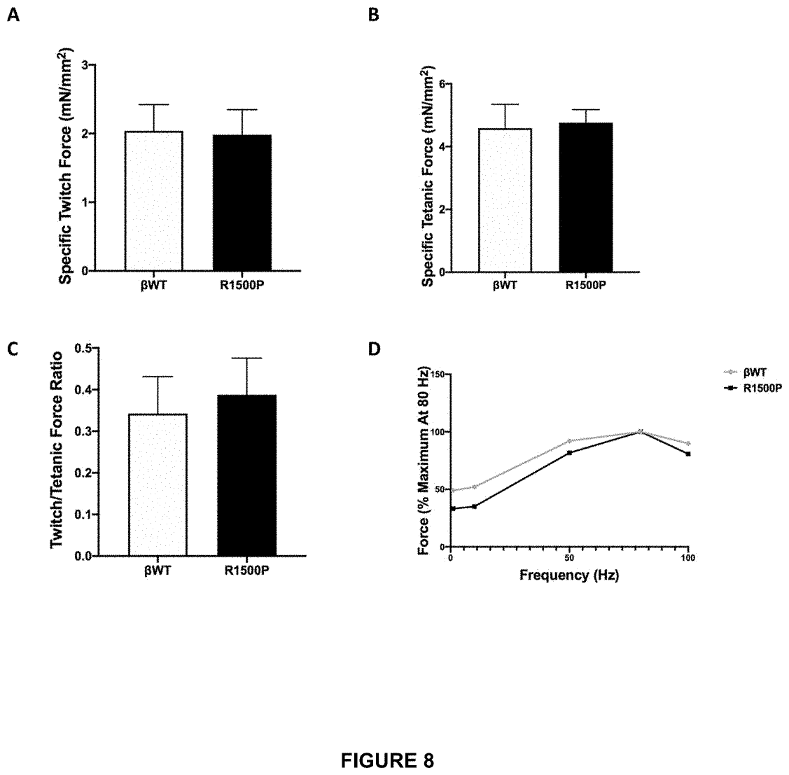

Since the parameters of muscle contractility, force, fatigability, and contractile kinetics can be measured in isolated muscles, the present inventors next assessed the ex-vivo properties of the extensor digitorum longus (EDL) muscles expressing βWT or R1500P myosins. EDL muscles were used instead of the TA due to the protocol requiring intact muscle-tendon complexes (30). As a control, the properties of the slow-twitch soleus muscle were also analyzed. Measurements of tetanic and twitch force were performed after determining the optimal muscle length. While specific tetanic force was not affected by the presence of the R1500P mutation (FIG. 5A), specific twitch force was significantly increased in R1500P EDL muscles while the twitch to tetanus force ratio was significantly decreased (FIG. 5B, 5C). In contrast, the activity of WT and R1500P soleus muscles, which do not express the transgene, did not show any functional difference (FIG. 8 A-D). FIG. 5D shows the force versus frequency relationship for EDL muscles obtained from WT and R1500P EDL muscles. In agreement with the observed decreased twitch/tetanus ratio, the R1500P curve is shifted to the right (downward). Notably, this phenotype, previously characterized as low-frequency fatigue, appears to be linked to altered calcium release. The present inventors next determined muscle fatigue by challenging the muscles with high frequency stimuli (100 Hz), which induced sustained muscle contraction; as the muscle relaxed over time, the force output as an indicator of fatigue was measured. The measurements obtained showed that the percentage of peak tetanic force dropped more quickly for R1500P muscle (FIG. 5E) suggesting that in addition to a drop in force production, the muscles expressing the mutant myosin have decreased resistance to fatigue.

### Example 6. The R1500P Mutation Affects Myofibril Relaxation

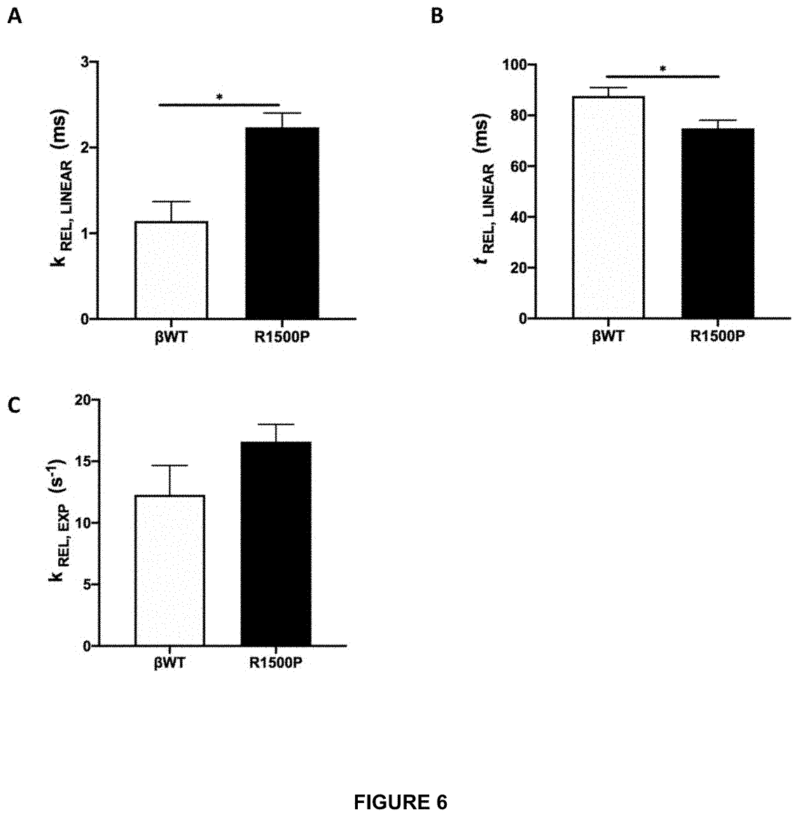

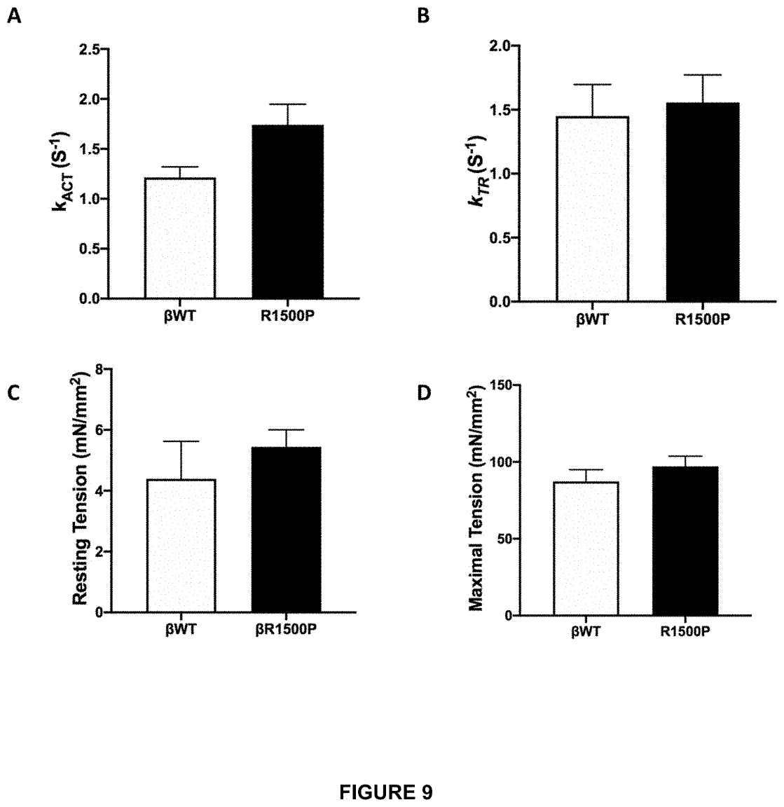

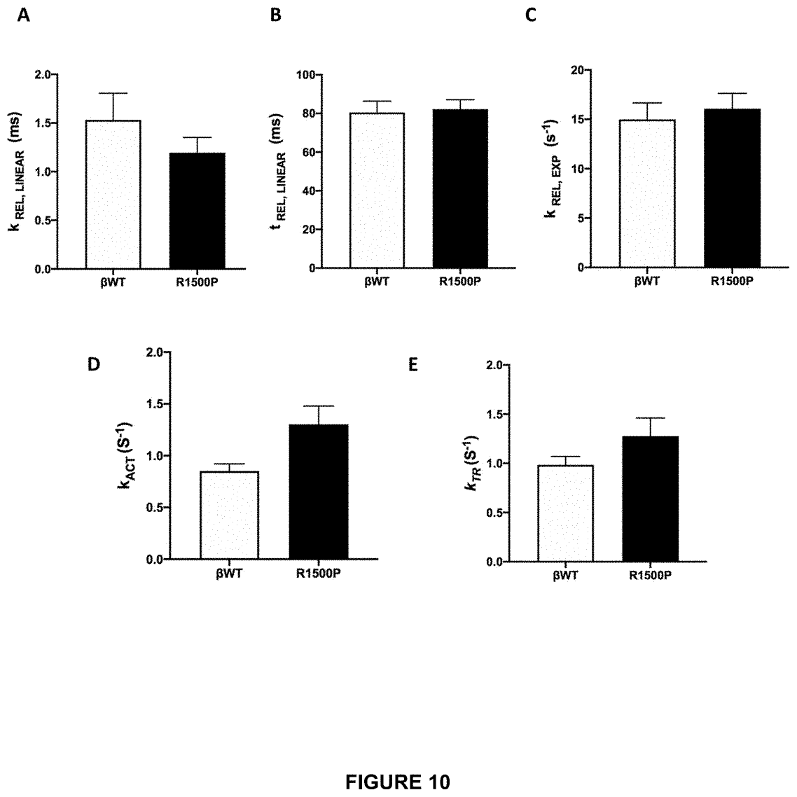

To delve more deeply into the whole animal and whole muscle phenotypes of R1500P mutant mice, the present inventors next analyzed force generation and relaxation kinetics of isolated myofibrils from TA muscle. Mounted myofibrils were activated and relaxed by rapidly switching between two flowing solutions of pCa 4.5 and pCa 9.0. After activation, rapid deactivation of myofibrils follows a biphasic state: an initial slow linear decay precedes a faster exponential decay. The rate of slow phase relaxation mirrors crossbridge detachment rate, whereas the duration of slow phase relaxation depends on Ca2+ activation levels. Although, no changes in any mechanical parameters were detected, which include resting, maximal tension, and activation kinetics, kACT or kTR. (FIG. 9 AD), the present inventors observed that the rate of slow phase relaxation (k_{rel, linear}) was significantly increased in R1500P myofibrils when compared to βWT controls (FIG. 6A), whereas the duration of slow phase relaxation (t_{rel, linear}) was significantly decreased (FIG. 6B). In contrast, the rate of the rapid exponential phase of relaxation (k_{rel, exp}) was unchanged (FIG. 6C). No difference in activation or relaxation was identified in myofibrils purified from βWT and R1500P soleus muscle (FIG. 10 A-E). The faster cross-bridge detachment observed in these experiments conducted on TA indicates that the proline mutation could cause muscle dysfunction muscle by affecting proper acto-myosin binding.

### Example 7. Materials and Methods

Animal Care: All animal experiments were performed using protocols approved by University of Colorado Institutional Animal Care and Use Committees (IACUC). Animals were housed under standard conditions in a partial barrier facility and received access to water and chow ad libitum. For sample collection, animals were sedated using 14% inhaled isoflurane and sacrificed by cervical dislocation. All data shown is from male mice.

Western Blotting: Protein lysates were prepared by homogenizing hindlimb muscle tissue in myosin extraction buffer (0.3M NaCl, 0.1M NaH2PO4, 0.05M Na2HPO4, 0.001M MgCl2.6H2O, 0.01M EDTA) following standard procedures. The antibodies used were against Myc-Tag (9B11) (1:10000, Cell Signaling Technology, #2276), F59 (1:2000), and α-sarcomeric actin (1:2000). All blots were imaged using the ImageQuant LAS 4000 (GE Healthcare Bio-Sciences, Pittsburgh, Pa.) system and analyzed with the ImageQuant software and/or with ImageJ.

RNA Isolation & Quantification: Total RNA was purified from hindlimb muscles using TRI Reagent (Ambion) according to manufacturer's protocol. cDNA was synthesized using SuperScript III reverse transcriptase (Invitrogen) and random hexamer primers. Gene expression was determined by qRT-PCR using SYBR Green dye (Invitrogen) and gene specific primer sets. All genes were normalized to 18S expression. Data were collected and analyzed using Bio-Rad CFX Real-Time PCR system.

Histology (Succinate Dehydrogenase Staining): Tibialis anterior muscles were snap frozen in isopentane/liquid N2, cryo-sectioned, and stained for enzymatic activities using standard procedures. The stained fibers were counted and their percentage of total number of fibers was calculated (150-200 total fibers/image, 3 images/mouse, 2 mice/genotype). Cross sectional area was determined using ImageJ.

Transmission Electron Microscopy: Skeletal muscle was dissected and immersed in 2.5% glutaraldehyde and 2% paraformaldehyde in 0.1 M cacodylate buffer at pH 7.4 for a minimum of 24 hours at 4° C. For processing, the tissue was rinsed in 100 mM cacodylate buffer and then immersed in 1% osmium and 1.5% potassium ferrocyanide for 15 min. Next, the tissue was rinsed five times in cacodylate buffer, immersed in 1% osmium for 1 hour, and then rinsed again five times for 2 min each in cacodylate buffer and two times briefly in water. The tissue was stained en bloc with 2% uranyl acetate for 1 hour before it was transferred to graded ethanols (50, 70, 90, and 100%) for 15 minutes each. Finally, the tissue was transferred through propylene oxide at room temperature and then embedded in LX112 and cured for 48 h at 60° C. in an oven. Ultra-thin sections (55 nm) were cut on a Reichert Ultracut S from a small trapezoid positioned over the tissue and were picked up on Formvar-coated slot grids or copper mesh grids (EMS). Sections were imaged on a FEI Tecnai G2 transmission electron microscope (Hillsboro, Oreg.) with an AMT digital camera (Woburn, Mass.).

Voluntary Wheel Running: Male mice were subjected to voluntary wheel running for a period of 28 days at the age of 3 months, 8 months, and 12 months. Mice were housed individually in a large cage with a running wheel. Exercise time, velocity, and distance were recorded daily for each animal.

Grip Strength: Forelimb grip strength was measured with a grip strength meter. The mice were first acclimated to the apparatus for approximately 5 min. Individual mice were then allowed to grab the bar while being held from the tip of their tail. The mouse was gently pulled away from the grip bar. When the mouse could no longer grasp the bar, the reading was recorded. Protocol was repeated five times with at least 30 sec rest between trials. The highest three values were averaged to obtain the absolute grip strength.

Four Limb Hanging Test: Male mice were placed in the center of a wire mesh screen; a timer was started and the screen was rotated to an inverted position with the mouse's head declining first. The screen was held above a padded surface. Either the time when the mouse falls was noted or the mouse was removed when the criterion time of 60 sec was reached.

Ex-Vivo Contractility Assay: Mice were euthanized according to NIH guidelines and IACUC institutional animal protocols. Extensor digitorum longus (EDL) was carefully dissected in total from the ligamentary attachment at the lateral condyle of the tibia to the insertion region. The muscle was transferred to a dish containing ice-cold isotonic physiologic salt solution (Tyrode's buffer (mM): NaCl 118, KCl 4, MgSO4 1.2, NaHCO₃25, NaH2PO4 1.2, glucose 10 and CaCl2) 2.5) bubbled with 95% O2/5% CO2 to maintain a pH of 7.4. The soleus was identified after removing the gastrocnemius muscle and was removed by cutting the ligaments connecting to the proximal half of the posterior tibia to the insertion, where the calcaneal tendon was cut and the muscle was placed into ice-cold Tyrode's buffer. Muscles were mounted vertically in individual tissue bath chambers and maintained at 37° C. Muscles were stretched and optimal length was set for each muscle. Stimulatory trains of varying frequency (1-100 Hz) were used to generate force-frequency curves. Tetanic force was achieved in all muscles using 100 Hz.

Myofibril Isolation: Myofibrils were isolated from flash frozen soleus and tibialis anterior as described (35, 36). A small section of muscle was cut into thin slices and bathed in 0.05% Triton X-100 in Linke's solution (132 mM NaCl, 5 mM KCl, 1 mM MgCl2, 10 mM Tris, 5 mM EGTA, 1 mM NaN3, pH 7.1) with protease inhibitor cocktail (10 μM leupeptin, 5 μM pepstatin, 200 μM phenyl-methylsuphonylfluoride, 10 μM E64, 500 μM NaN3, 2 mM dithioerythritol) overnight at 4° C. overnight. Skinned tissue was washed three times in rigor solution (50 mM Tris, 100 mM KCl, 2 mM MgCl2, 1 mM EGTA, pH 7.0) and resuspended in bath solution with protease inhibitors (pCa 9.0; 100 mM Na2EGTA; 1M potassium propionate; 100 mM Na2SO4; 1M MOPS; 1M MgCl2; 6.7 mM ATP; and 1 mM creatine phosphate; pH 7.0) and homogenized at medium speed for 10 seconds three times.

Myofibril Mechanics: Myofibrils were isolated and mechanical parameters were measured as described (36-38). Myofibrils were placed on a glass coverslip in relaxing solution at 15° C. and then a small bundle of myofibrils was mounted on two microtools. One microtool was attached to a motor that produces rapid length changes (Mad City Labs) and the second microtool was a calibrated cantilevered force probe (5.8 μm/N; frequency response 2-5 KHz). Myofibril length was set at 5-10% above slack length and average sarcomere length and myofibril diameter were measured using ImageJ. Mounted myofibrils were activated and relaxed by rapidly translating the interface between two flowing streams of solutions of pCa 4.5 and pCa 9.0 (38, 39). Data was collected and analyzed using customized LabView software. Measured mechanical and kinetic parameters were defined as follows: resting tension (mN/mm2)—myofibril basal tension in fully relaxing condition; maximal tension (mN/mm2)—maximal tension generated at full calcium activation (pCa 4.5); the rate constant of tension development following maximal calcium activation (kACT); the rate constant of tension redevelopment following a release-restretch applied to the activated myofibril (kTR) (40); rate constant of early slow force decline (kREL, LIN)—the slope of the linear regression normalized to the amplitude of relaxation transient, duration of early slow force decline—measured from onset of solution change to the beginning of the exponential force decay, the rate constant of the final exponential phase of force decline (kREL, EXP).

Data & Statistical Analyses: Data are presented as mean±SEM. Differences between groups were evaluated for statistical significance using Student's two-tailed t test (two groups) or one-way ANOVA (more than two groups) followed by Tukey's post-hoc test for pairwise comparisons. P values less than 0.05 were considered significant unless otherwise noted.

### REFERENCES

1. Muelas N, et al. (2010) MYH7 gene tail mutation causing myopathic profiles beyond Laing distal myopathy. Neurology 75(8):732-741.
2. Laing N G, et al. (1995) Autosomal dominant distal myopathy: linkage to chromosome 14. Am J Hum Genet 56(2):422-427.
3. Mastaglia F L, et al. (2002) Early onset chromosome 14-linked distal myopathy (Laing). Neuromuscular Disorders 12(4):350-357.
4. Jandreski M A, Sole M J, Liew C C (1987) Two different forms of beta myosin heavy chain are expressed in human striated muscle. Hum Genet 77(2):127-131.
5. Hedera P, Petty E M, Bui M R, Blaivas M, Fink J K (2003) The Second Kindred With Autosomal Dominant Distal Myopathy Linked to Chromosome 14q: Genetic and Clinical Analysis. Arch Neurol 60(9):1321-1325.
6. Udd B (2009) 165th ENMC International Workshop: Distal myopathies 6-8th February 2009 Naarden, The Netherlands. Neuromuscular Disorders 19(6):429-438.
7. Lamont P J, et al. (2014) Novel Mutations Widen the Phenotypic Spectrum of Slow Skeletal/-Cardiac Myosin (MYH7) Distal Myopathy. HumanMutation 35(7):868-879.
8. Kärkkäinen S, et al. (2004) Two novel mutations in the β-myosin heavy chain gene associated with dilated cardiomyopathy. European Journal of Heart Failure 6(7):861-868.
9. Meredith C, et al. (2004) Mutations in the Slow Skeletal Muscle Fiber Myosin Heavy Chain Gene (MYH7) Cause Laing Early-Onset Distal Myopathy (MPD1). The American Journal of Human Genetics 75(4):703-708.
10. Dubourg O, et al. (2011) A novel MYH7 mutation occurring independently in French and Norwegian Laing distal myopathy families and de novo in one Finnish patient. J Neurol 258(6):1157-1163.
11. Darin N, Tajsharghi H, Ostman-Smith I, Gilljam T, Oldfors A (2007) New skeletal myopathy and cardiomyopathy associated with a missense mutation in MYH7. Neurology 68(23):2041-2042.
12. Homayoun H, et al. (2011) Novel mutation in MYH7 gene associated with distal myopathy and cardiomyopathy. Neuromuscular Disorders 21(3):219-222.
13. Barlow D J, Thornton J M (1988) Helix geometry in proteins. Journal of Molecular Biology 201(3):601-619.
14. Buvoli M, Buvoli A, Leinwand L A (2012) Effects of Pathogenic Proline Mutations on Myosin Assembly. J Mol Biol 415(5):807-818.
15. Dahl-Halvarsson M, Pokrzywa M, Rauthan M, Pilon M, Tajsharghi H (2017) Myosin Storage Myopathy in C. elegans and Human Cultured Muscle Cells. PLoS One 12(1). doi:10.1371/journal.pone.0170613.
16. Parker F, et al. (2018) A1603P and K1617del, Mutations in β-Cardiac Myosin Heavy Chain that Cause Laing Early-Onset Distal Myopathy, Affect Secondary Structure and Filament Formation In Vitro and In Vivo. J Mol Biol 430(10):1459-1478.
17. Murgiano L, Tammen I, Harlizius B, Drögemüller C (2012) A de novo germline mutation in MYH7 causes a progressive dominant myopathy in pigs. BMC Genet 13:99.
18. Dahl-Halvarsson M, et al. (2018) Drosophila model of myosin myopathy rescued by overexpression of a TRIM-protein family member. PNAS 115(28):E6566-E6575.
19. Maass A, Leinwand L (2000) Animal models of hypertrophic cardiomyopathy. Current Opinion in Cardiology 15(3):189-196.
20. Tardiff J C, et al. (2000) Expression of the R (slow)-isoform of MHC in the adult mouse heart causes dominant-negative functional effects. American Journal of Physiology-Heart and Circulatory Physiology 278(2):H412-H419.
21. Dunant P, et al. (2003) Expression of dystrophin driven by the 1.35-kb MCK promoter ameliorates muscular dystrophy in fast, but not in slow muscles of transgenic mdx mice. Molecular Therapy 8(1):80-89.
22. Lamont P J, et al. (2006) Laing early onset distal myopathy: slow myosin defect with variable abnormalities on muscle biopsy. J Neurol Neurosurg Psychiatry 77(2):208-215.
23. Rayavarapu S, Coley W, Nagaraju K (2012) Endoplasmic Reticulum Stress in Skeletal Muscle Homeostasis and Disease. Curr Rheumatol Rep 14(3):238-243.
24. Wu J, Kaufman R J (2006) From acute ER stress to physiological roles of the Unfolded Protein Response. Cell Death Differ 13(3):374-384.
25. Mollereau B, Manie S, Napoletano F (2014) Getting the better of ER stress. J Cell Commun Signal 8(4):311-321.
26. Rashid H-O, Yadav R K, Kim H-R, Chae H-J (2015) ER stress: Autophagy induction, inhibition and selection. Autophagy 11(11):1956-1977.
27. Hulmi J J, et al. (2016) Effects of muscular dystrophy, exercise and blocking activin receptor IIB ligands on the unfolded protein response and oxidative stress. Free Radic Biol Med 99:308-322.
28. Moorwood C, Barton E R (2014) Caspase-12 ablation preserves muscle function in the mdx mouse. Hum Mol Genet 23(20):5325-5341.
29. Pauly M, et al. (2017) ER stress disturbs SR/ER-mitochondria Ca2+ transfer: Implications in Duchenne muscular dystrophy. Biochim Biophys Acta Mol Basis Dis 1863(9):2229-2239.
30. Ikezoe K, et al. (2007) Endoplasmic reticulum stress in myotonic dystrophy type 1 muscle. Acta Neuropathol 114(5):527-535.
31. Park K H, et al. (2012) Ex Vivo Assessment of Contractility, Fatigability and Alternans in Isolated Skeletal Muscles. J Vis Exp (69). doi:10.3791/4198.
32. Edwards R H, Hill D K, Jones D A, Merton P A (1977) Fatigue of long duration in human skeletal muscle after exercise. J Physiol 272(3):769-778.
33. MacArthur M W, Thornton J M (1991) Influence of proline residues on protein conformation. Journal of Molecular Biology 218(2):397-412.
34. Al-Qusairi L, Laporte J (2011) T-tubule biogenesis and triad formation in skeletal muscle and implication in human diseases. Skelet Muscle 1:26.
35. Westerblad H, Bruton J D, Allen D G, Lännergren J (2000) Functional significance of Ca2+ in long-lasting fatigue of skeletal muscle. Eur J Appl Physiol 83(2):166-174.
36. Jeong M Y, et al. (2018) Histone deacetylase activity governs diastolic dysfunction through a nongenomic mechanism. Sci Transl Med 10(427). doi:10.1126/scitranslmed.aao0144.
37. Woulfe K C, et al. (2019) A Novel Method of Isolating Myofibrils From Primary Cardiomyocyte Culture Suitable for Myofibril Mechanical Study. Front Cardiovasc Med 6. doi:10.3389/fcvm.2019.00012.
38. Colomo F, Nencini S, Piroddi N, Poggesi C, Tesi C (1998) Calcium dependence of the apparent rate of force generation in single striated muscle myofibrils activated by rapid solution changes. Adv Exp Med Biol 453:373-381; discussion 381-382.
39. Tesi C, Colomo F, Nencini S, Piroddi N, Poggesi C (2000) The Effect of Inorganic Phosphate on Force Generation in Single Myofibrils from Rabbit Skeletal Muscle. Biophysical Journal 78(6):3081-3092.
40. Tesi C, Colomo F, Nencini S, Piroddi N, Poggesi C (1999) Modulation by substrate concentration of maximal shortening velocity and isometric force in single myofibrils from frog and rabbit fast skeletal muscle. J Physiol 516(Pt 3):847-853.
41. Brenner B (1988) Effect of Ca2+ on cross-bridge turnover kinetics in skinned single rabbit psoas fibers: implications for regulation of muscle contraction. Proc Natl Acad Sci USA 85(9):3265-3269.

SEQUENCE IDENTIFICATION
Amino Acid
MYH7 R1500P
Mus musculus
SEQ ID NO. 1
MADAEMAAFGAAAPFLRKSEKERLEAQTRPFDLKKDVFVPDDKEEFVKAKIVSREGGKVTAETENGKTVTVKEDQVM
QQNPPKFDKIEDMAMLTFLHEPAVLYNLKERYASWMIYTYSGLFCVTVNPYKWLPVYNAEVVAAYRGKKRSEAPPHI
FSISDNAYQYMLTDRENQSILITGESGAGKTVNTKRVIQYFAVIAAIGDRSKKDQTPGKGTLEDQIIQANPALEAFG
NAKTVRNDNSSREGKEIRIHFGATGKLASADIETYLLEKSRVIFQLKAERDYHIFYQILSNKKPELLDMLLITNNPY
DYAFISQGETTVASIDDSEELMATDSAFDVLGFTPEEKNSIYKLTGAIMHEGNMKEKQKQREEQAEPDGTEEADKSA
YLMGLNSADLLKGLCHPRVKVGNEYVTKGQNVQQVSYAIGALAKSVYEKMENWMVTRINATLETKQPRQYFIGVLDI
AGFEIEDENSFEQLCINFTNEKLQQFFNHHMFVLEQEEYKKEGIEWTFIDFGMDLQACIDLIEKPMGIMSILEEECM
FPKATDMTFKAKLYDNHLGKSNNFQKPRNVKGKQEAHFSLVHYAGTVDYNILGWLQKNKDPLNETVVGLYQKSSLKL
LSNLFANYAGADAPADKGKGKAKKGSSFQTVSALHRENLNKLMTNLRSTHPHFVRCIIPNETKSPGVMDNPLVMHQL
RCNGVLEGIRICRKGFPNRILYGDFRQRYRILNPAAIPEGQFIDSRKGAEKLLGSLDIDHNQYKFGHTKVFFKAGLL
GLLEEMRDERLSRIITRIQAQSRGVLSRMEFKKLLERRDSLLIIQWNIRAFMGVKNWPWMKLYFKIKPLLKSAETEK
EMATMKEEFGRVKDALEKSEARRKELEEKMVSLLQEKNDLQLQVQAEQDNLADAEERCDQLIKNKIQLEAKVKEMTE
RLEDEEEMNAELTAKKRKLEDECSELKRDIDDLELTLAKVEKEKHATENKVKNLTEEMAGLDEIIVKLTKEKKALQE
AHQQALDDLQAEEDKVNTLTKAKVKLEQQVDDLEGSLEQEKKVRMDLERAKRKLEGDLKLTQESIMDLENDKQQLDE
RLKKKDFELNALNARIEDEQALGSQLQKKLKELQARIEELEEELEAERTARAKVEKLRSDLSRELEEISERLEEAGG
ATSVQIEMNKKREAEFQKMRRDLEEATLQHEATAAALRKKHADSVAELGEQIDNLQRVKQKLEKEKSEFKLELDDVT
SNMEQIIKAKANLEKMCRTLEDQMNEHRSKAEETQRSVNDLTSQRAKLQTENGELSRQLDEKEALISQLTRGKLTYT
QQLEDLKRQLEEEVKAKNALAHALQSARHDCDLLREQYEEETEAKAELQRVLSKANSEVAQWRTKYETDAIQRTEEL
EEAKKKLAQRLQDAEEAVEAVNAKCSSLEKTKHRLQNEIEDLMVDVERSNAAAAALDKKQRNFDKILAEWKQKYEES
QSELESSQKEARSLSTELFKLKNAYEESLEHLETFKPENKNLQEEISDLTEQLGSTGKSIHELEKIRKQLEAEKLEL
QSALEEAEASLEHEEGKILRAQLEFNQIKAEIERKLAEKDEEMEQAKRNHLRMVDSLQTSLDAETRSRNEALRVKKK
MEGDLNEMEIQLSHANRMAAEAQKQVKSLQSLLKDTQIQLDDAVRANDDLKENIAIVERRNNLLQAELEELRAVVEQ
TERSRKLAEQELIETSERVQLLHSQNTSLINQKKKMDADLSQLQTEVEEAVQECRNAEEKAKKAITDAAMMAEELKK
EQDTSAHLERMKKNMEQTIKDLQHRLDEAEQIALKGGKKQLQKLEARVRELENELEAEQKRNAESVKGMRKSERRIK
ELTYQTEEDRKNLLRLQDLVDKLQLKVKAYKRQAEEAEEQANTNLSKFRKVQHELDEAEERADIAESQVNKLRAKSR
DIGAKGLNEE
Amino Acid
MYH7-WT
Mus musculus
SEQ ID NO. 2
MADAEMAAFGAAAPFLRKSEKERLEAQTRPFDLKKDVFVPDDKEEFVKAKIVSREGGKVTAETENGKTVTVKEDQVM
QQNPPKFDKIEDMAMLTFLHEPAVLYNLKERYASWMIYTYSGLFCVTVNPYKWLPVYNAEVVAAYRGKKRSEAPPHI
FSISDNAYQYMLTDRENQSILITGESGAGKTVNTKRVIQYFAVIAAIGDRSKKDQTPGKGTLEDQIIQANPALEAFG
NAKTVRNDNSSREGKEIRIHFGATGKLASADIETYLLEKSRVIFQLKAERDYHIFYQILSNKKPELLDMLLITNNPY
DYAFISQGETTVASIDDSEELMATDSAFDVLGFTPEEKNSIYKLTGAIMHEGNMKEKQKQREEQAEPDGTEEADKSA
YLMGLNSADLLKGLCHPRVKVGNEYVTKGQNVQQVSYAIGALAKSVYEKMENWMVTRINATLETKQPRQYFIGVLDI
AGFEIEDENSFEQLCINFTNEKLQQFFNHHMFVLEQEEYKKEGIEWTFIDFGMDLQACIDLIEKPMGIMSILEEECM
FPKATDMTFKAKLYDNHLGKSNNFQKPRNVKGKQEAHFSLVHYAGTVDYNILGWLQKNKDPLNETVVGLYQKSSLKL
LSNLFANYAGADAPADKGKGKAKKGSSFQTVSALHRENLNKLMTNLRSTHPHFVRCIIPNETKSPGVMDNPLVMHQL
RCNGVLEGIRICRKGFPNRILYGDFRQRYRILNPAAIPEGQFIDSRKGAEKLLGSLDIDHNQYKFGHTKVFFKAGLL
GLLEEMRDERLSRIITRIQAQSRGVLSRMEFKKLLERRDSLLIIQWNIRAFMGVKNWPWMKLYFKIKPLLKSAETEK
EMATMKEEFGRVKDALEKSEARRKELEEKMVSLLQEKNDLQLQVQAEQDNLADAEERCDQLIKNKIQLEAKVKEMTE
RLEDEEEMNAELTAKKRKLEDECSELKRDIDDLELTLAKVEKEKHATENKVKNLTEEMAGLDEIIVKLTKEKKALQE
AHQQALDDLQAEEDKVNTLTKAKVKLEQQVDDLEGSLEQEKKVRMDLERAKRKLEGDLKLTQESIMDLENDKQQLDE
RLKKKDFELNALNARIEDEQALGSQLQKKLKELQARIEELEEELEAERTARAKVEKLRSDLSRELEEISERLEEAGG
ATSVQIEMNKKREAEFQKMRRDLEEATLQHEATAAALRKKHADSVAELGEQIDNLQRVKQKLEKEKSEFKLELDDVT
SNMEQIIKAKANLEKMCRTLEDQMNEHRSKAEETQRSVNDLTSQRAKLQTENGELSRQLDEKEALISQLTRGKLTYT
QQLEDLKRQLEEEVKAKNALAHALQSARHDCDLLREQYEEETEAKAELQRVLSKANSEVAQWRTKYETDAIQRTEEL
EEAKKKLAQRLQDAEEAVEAVNAKCSSLEKTKHRLQNEIEDLMVDVERSNAAAAALDKKQRNFDKILAEWKQKYEES
QSELESSQKEARSLSTELFKLKNAYEESLEHLETFKRENKNLQEEISDLTEQLGSTGKSIHELEKIRKQLEAEKLEL
QSALEEAEASLEHEEGKILRAQLEFNQIKAEIERKLAEKDEEMEQAKRNHLRMVDSLQTSLDAETRSRNEALRVKKK
MEGDLNEMEIQLSHANRMAAEAQKQVKSLQSLLKDTQIQLDDAVRANDDLKENIAIVERRNNLLQAELEELRAVVEQ
TERSRKLAEQELIETSERVQLLHSQNTSLINQKKKMDADLSQLQTEVEEAVQECRNAEEKAKKAITDAAMMAEELKK
EQDTSAHLERMKKNMEQTIKDLQHRLDEAEQIALKGGKKQLQKLEARVRELENELEAEQKRNAESVKGMRKSERRIK
ELTYQTEEDRKNLLRLQDLVDKLQLKVKAYKRQAEEAEEQANTNLSKFRKVQHELDEAEERADIAESQVNKLRAKSR
DIGAKGLNEE
Amino Acid
MYH7
Homo sapiens
SEQ ID NO. 3
MGDSEMAVFGAAAPYLRKSEKERLEAQTRPFDLKKDVFVPDDKQEFVKAKIVSREGGKVTAETEYGKTVTVKEDQVM
QQNPPKFDKIEDMAMLTFLHEPAVLYNLKDRYGSWMIYTYSGLFCVTVNPYKWLPVYTPEVVAAYRGKKRSEAPPHI
FSISDNAYQYMLTDRENQSILITGESGAGKTVNTKRVIQYFAVIAAIGDRSKKDQSPGKGTLEDQIIQANPALEAFG
NAKTVRNDNSSREGKEIRIHFGATGKLASADIETYLLEKSRVIFQLKAERDYHIFYQILSNKKPELLDMLLITNNPY
DYAFISQGETTVASIDDAEELMATDNAFDVLGFTSEEKNSMYKLTGAIMHEGNMKFKLKQREEQAEPDGTEEADKSA
YLMGLNSADLLKGLCHPRVKVGNEYVTKGQNVQQVIYATGALAKAVYERMFNWMVTRINATLETKQPRQYFIGVLDI
AGFEIEDENSFEQLCINFTNEKLQQFFNHHMFVLEQEEYKKEGIEWTFIDEGMDLQACIDLIEKPMGIMSILEEECM
FPKATDMTFKAKLFDNHLGKSANFQKPRNIKGKPEAHFSLIHYAGIVDYNIIGWLQKNKDPLNETVVGLYQKSSLKL
LSTLFANYAGADAPIEKGKGKAKKGSSFQTVSALHRENLNKLMTNLRSTHPHFVRCIIPNETKSPGVMDNPLVMHQL
RCNGVLEGIRICRKGFPNRILYGDFRQRYRILNPAAIPEGQFIDSRKGAEKLLSSLDIDHNQYKFGHTKVFFKAGLL
GLLEEMRDERLSRIITRIQAQSRGVLARMEYKKLLERRDSLLVIQWNIRAFMGVKNWPWMKLYFKIKPLLKSAEREK
EMASMKEEFTRLKEALEKSEARRKELEEKMVSLLQEKNDLQLQVQAEQDNLADAEERCDQLIKNKIQLEAKVKEMNE
RLEDEEEMNAELTAKKRKLEDECSELKRDIDDLELTLAKVEKEKHATENKVKNLTEEMAGLDEIIAKLTKEKKALQE
AHQQALDDLQAEEDKVNTLTKAKVKLEQQVDDLEGSLEQEKKVRMDLERAKRKLEGDLKLTQESIMDLENDKQQLDE
RLKKKDFELNALNARIEDEQALGSQLQKKLKELQARIEELEEELEAERTARAKVEKLRSDLSRELEEISERLEEAGG
ATSVQIEMNKKREAEFQKMRRDLEEATLQHEATAAALRKKHADSVAELGEQIDNLQRVKQKLEKEKSEFKLELDDVT
SNMEQIIKAKANLEKMCRTLEDQMNEHRSKAEETQRSVNDLTSQRAKLQTENGELSRQLDEKEALISQLTRGKLTYT
QQLEDLKRQLEEEVKAKNALAHALQSARHDCDLLREQYEEETEAKAELQRVLSKANSEVAQWRTKYETDAIQRTEEL
EEAKKKLAQRLQEAEEAVEAVNAKCSSLEKTKHRLQNEIEDLMVDVERSNAAAAALDKKQRNFDKILAEWKQKYEES
QSELESSQKEARSLSTELFKLKNAYEESLEHLETFKRENKNLQEEISDLTEQLGSSGKTIHELEKVRKQLEAEKMEL
QSALEEAEASLEHEEGKILRAQLEFNQIKAEIERKLAEKDEEMEQAKRNHLRVVDSLQTSLDAETRSRNEALRVKKK
MEGDLNEMEIQLSHANRMAAEAQKQVKSLQSLLKDTQIQLDDAVRANDDLKENIAIVERRNNLLQAELEELRAVVEQ
TERSRKLAEQELIETSERVQLLHSQNTSLINQKKKMDADLSQLQTEVEEAVQECRNAEEKAKKAITDAAMMAEELKK
EQDTSAHLERMKKNMEQTIKDLQHRLDEAEQIALKGGKKQLQKLEARVRELENELEAEQKRNAESVKGMRKSERRIK
ELTYQTEEDRKNLLRLQDLVDKLQLKVKAYKRQAEEAEEQANTNLSKFRKVQHELDEAEERADIAESQVNKLRAKSR
DIGTKGLNEE

What is claimed is:

1. A Laing distal myopathy (MPD1) model animal expressing a β-myosin R1500P mutant transgene wherein the arginine (R) residue at amino acid position 1500 is substituted with a proline (P) residue, and wherein said transgene causes at least one pathological phenotype associated with MPD1.

2. The MPD1 model animal of claim 1, wherein said animal is selected from the group consisting of: a rodent, and a mouse.

3. The MPD1 model animal of claim 1, and further comprising wherein the wild-type β-myosin has been knocked-out or its expression disrupted.

4. The MPD1 model animal of claim 1, wherein said β-myosin R1500P mutant transgene comprises the nucleotide sequence encoding the amino acid sequence according to SEQ ID NO. 1, or a fragment or variant thereof.

5. The MPD1 model animal of claim 1, wherein said β-myosin R1500P mutant transgene is operably linked to a promoter selected from the group consisting of: an inducible promoter, a tissue-specific promoter, and a muscle creatine kinase (MCK) promoter.

6. The MPD1 model animal of claim 1, further comprising a tag coupled with said β-myosin R1500P mutant transgene.

7. The MPD1 model animal of claim 6, wherein said tag comprises a myc-tag.

8. The MPD1 model animal of claim 1, wherein the pathological phenotype associated with MPD1 is selected from the group consisting of:
abnormal muscle tissue or muscle atrophy;
decreased the muscle/body weight ratio;
muscle tissue had a higher proportion of smaller muscle fibers;
upregulation of expression of myosin isoforms Myh7 and Myh4;
abnormalities in sarcoplasmic reticulum (SR);
abnormalities in t-tubules;
abnormalities in mitochondria;
upregulation of one or more gene of the unfolded protein response (UPR) pathway;
upregulation of one or more gene of the PERK, or genes involved in the PERK pathway;
upregulation of ATF4;
upregulation of ATF3;
upregulation of GADD34;
decreased muscle strength;
decreased resistance to fatigue; and
weakened actomyosin binding.

9. The transgenic, non-human animal whose genome comprises a β-myosin R1500P mutant transgene wherein the arginine (R) residue at amino acid position 1500 is substituted with a proline (P) residue.

10. The transgenic animal of claim 9, wherein said animal is selected from the group consisting of: a rodent, and a mouse.

11. The transgenic animal of any of claim 9, wherein said wild-type β-myosin gene comprises the nucleotide sequence encoding the amino acid sequence according to SEQ ID NO. 2, or a fragment or variant thereof.

12. The transgenic animal of any of claim 9, wherein said β-myosin R1500P mutant comprises the nucleotide sequence encoding the amino acid sequence according to SEQ ID NO. 1, or a fragment or variant thereof.

13. The transgenic animal of any of claim 9, wherein said β-myosin R1500P mutant transgene is operably linked to a promoter selected from the group consisting of: an inducible promoter, a tissue-specific promoter, and a muscle creatine kinase (MCK) promoter.

14. The transgenic animal of any of claim 9, further comprising a tag coupled with said β-myosin R1500P mutant transgene.

15. The transgenic animal of any of claim 9, wherein said transgenic animal exhibits at least one phenotype associated with Laing distal myopathy (MPD1).

16. The transgenic animal of claim 15, wherein the phenotype associated with MPD1 associated is selected from the group consisting of:
abnormal muscle tissue or muscle atrophy;
decreased the muscle/body weight ratio;
muscle tissue had a higher proportion of smaller muscle fibers;
upregulation of expression of myosin isoforms Myh7 and Myh4;
abnormalities in sarcoplasmic reticulum (SR);
abnormalities in t-tubules;
abnormalities in mitochondria;
upregulation of one or more gene of the unfolded protein response (UPR) pathway;
upregulation of one or more gene of the PERK, or genes involved in the PERK pathway;
upregulation of ATF4;
upregulation of ATF3;
upregulation of GADD34;
decreased muscle strength;
decreased resistance to fatigue; and
weakened actomyosin binding.

17. A modified non-human mammalian cell expressing a heterologous nucleotide, operably linked to a promoter, encoding a β-myosin R1500P mutant transgene according to SEQ ID NO. 1, or a fragment of variant thereof.

18. The cell of claim 17, wherein the expression of a wild type β-myosin gene of the cell has been deleted, or disrupted.

19. The cell of claim 17, wherein said cell is a rodent cell, or a mouse cell.

20. The cell of claim 17, wherein the cell is heterozygous for the modification, or wherein the cell is heterozygous for the modification.
